(19) United States
(12) Patent Application Publication
(10) Pub. No.: US 2008/0112853 A1
(43) Pub. Date: May 15, 2008

(54) METHOD AND APPARATUS FOR ANALYTE MEASUREMENTS IN THE PRESENCE OF INTERFERENTS

(76) Inventor: W. Dale Hall, Oakland, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/839,447

(22) Filed: Aug. 15, 2007

Related U.S. Application Data
(60) Provisional application No. 60/837,746, filed on Aug. 15, 2006.
(60) Provisional application No. 60/950,093, filed on Jul. 16, 2007.

(51) Int. Cl. G01N 21/01; B01J 19/00; A61B 5/145; G06F 19/00

(52) U.S. Cl. 422/82.05; 422/68.1; 600/309; 702/25

(57) ABSTRACT

Method and apparatus are described that permit an analyte concentration to be estimated from a measurement in the presence of compounds that interfere with the measurement. The method reduces the error in the analyte concentration in the presence of interferents. The method includes the use of a set of measurements obtained for a large population having a range of known analyte and interfering compound concentrations. From a sample measurement, which may or may not be one of the population, interferents likely to be present are identified, and a calibration coefficient is calculated. The calibration coefficient may be applied to the measurement to estimate the analyte concentration. In some implementations, the calibration coefficient may be determined as a weighted average of single interferent calibration coefficients. In some embodiments, the sample measurement includes a spectroscopic measurement.

### CROSS-REFERENCE TO RELATED APPLICATIONS

This application claims the benefit under 35 U.S.C. § 119(e) of U.S. Provisional Patent Application No. 60/837,746, filed Aug. 15, 2006, entitled “METHOD AND APPARATUS FOR ANALYTE MEASUREMENTS IN THE PRESENCE OF INTERFERENTS,” and U.S. Provisional Patent Application No. 60/950,093, filed Jul. 16, 2007, entitled “ANALYTE MEASUREMENT SYSTEMS AND METHODS,” all of which are hereby incorporated by reference in their entirety herein.

### BACKGROUND

1. Field

Certain embodiments disclosed herein relate to method and apparatus for determining the concentration of an analyte in a sample, and more particularly to method and apparatus that reduce error in determining the analyte concentration in the presence of sample components that interfere with the analyte measurement.

2. Description of the Related Art

Spectroscopic analysis is a powerful technique for determining the presence of one or more analytes in a sample by monitoring the interaction of light with the sample. Examples of spectroscopic measurements include, but are not limited to, the determination of the amount of light transmitted, absorbed, reflected, and/or scattered from a sample at one or more wavelengths. Thus, for example, absorption analysis includes determining the decrease in the intensity of light transmitted through a sample at one or more wavelengths, and then comparing the decrease in intensity with an absorption model based, for example, on Beer's law.

### SUMMARY

Various embodiments of the systems and methods disclosed herein provide reduced sensitivity for analyte estimation in the presence of interferents, so that, over the ranges of likely interferent concentrations, the net effect of the interferents on the analyte estimation is reduced below that of the sensitivity to an analyte of interest.

In some embodiments, method and apparatus are provided for determining an analyte concentration in a sample that may contain interferents. Possible interferents in the sample are determined by analysis of a sample measurement. In another embodiment, a calibration for estimating an analyte concentration in a sample is generated to minimize the error in the estimation due to possible interferents. In another embodiment, the analyte concentration is estimated from a sample measurement, a plurality of Sample Population spectra taken in the absence of interferents, and a library of interferent spectrum.

In some embodiments, a method is provided for estimating the amount of an analyte in a sample from a measurement, where the sample may include one or more interferents that affect the measurement. The method includes determining the presence of possible interferents to the estimation of the analyte concentration, and determining a calibration that reduces errors in the calibration due to the presence of the determined possible interferents.

One embodiment includes a method of spectroscopically identifying an interferent in a material sample. The method includes forming a statistical model of interferent-free spectra, comparing combinations of material sample spectra and interferent spectra corresponding to varying concentrations of the interferent, and identifying the interferent as a possible interferent if any of the combinations are within predetermined bounds.

One embodiment includes a method for estimating the amount of an analyte in a sample from a measurement of the sample. The method includes identifying one or more possible interferents to the measurement of the analyte in the sample, and calculating a calibration that, when applied to the measurement, provides an estimate of the analyte concentration in the sample. The calculation reduces or minimizes the error of interferents on the estimated analyte concentration.

One embodiment includes a method of generating an average calibration vector for estimating the amount of an analyte from the spectrum of a sample having one or more identified interferents. The method includes forming a plurality of spectra each including a combination of one of a plurality of interferent-free spectra, each having a known amount of analyte, and the spectrum of random combinations of possible amounts of the one or more interferents; forming a plurality of first subsets of spectra each including a random selection of the plurality of spectra and defining a corresponding second subset of spectra of the plurality of spectra not included in the first subset. For each first subset of spectra, the method further includes generating a calibration vector using the known analyte concentration corresponding to each spectrum, estimating the amount of analyte from each spectrum of the corresponding second subset using the generated calibration vector, and determining a subset-average error between the estimated amount of analyte and the known amount of analyte. The method further includes calculating an average calibration vector from the calibration vector and determined average error from each subset of spectra to reduce the variance of the error obtained by the use of the averaged calibration. In some embodiments of this method, the variance of the error is minimized using a mathematical minimization technique.

One embodiment includes a method of generating a calibration vector or estimating an analyte where the measurement is a spectrum. In one embodiment, the spectrum is an infrared spectrum, such as a near infrared and/or a mid infrared spectrum. In another embodiment, the measurement is obtained on a material sample from a person.

One embodiment includes a method to determine a calibration that minimizes errors in the calibration due to the presence of the determined possible interferents.

One embodiment includes a carrier medium carrying one or more computer readable code segments to instruct a processor to implement any one or combination of the methods disclosed herein. Other embodiments include a computer system programmed to carry out any one or combination of the methods disclosed herein.

One embodiment comprises a method of estimating the concentration of an analyte in a sample from a measurement, where the sample may include one or more interferents that affect the measurement. The method comprises determining the presence in the sample of possible interferents to the measurement, and determining a calibration that reduces errors in the measurement due to the presence of the determined possible interferents. The method can further comprise applying the calibration to the measurement, and estimating the analyte concentration based on the calibrated measurement. The measurement can be from a person, wherein the determining the presence of possible interferents and the determining a calibration both include comparing the measurement with population measurements, and where the determining does not require the population to include the person. The measurement can further comprise a spectrum obtained from a material sample, and the spectrum can be an infrared spectrum, such as a near infrared spectrum and/or a mid infrared spectrum. The measurement can also further comprise a spectrum obtained from a material sample non-invasively. The material sample can include at least one of the following: blood, a component of blood, interstitial fluid, or urine. The calibration can comprise a vector that is not required to be perpendicular to the spectra of the determined possible interferents. Determining a calibration can minimize errors in the calibration due to the presence of the determined possible interferents.

One embodiment comprises a carrier medium carrying one or more computer readable code segments to instruct a processor to implement a method of estimating the amount of an analyte in a sample from a measurement, where the sample may include one or more interferents that affect the measurement. The method comprises determining the presence in the sample of possible interferents to the measurement, and determining a calibration that reduces errors in the measurement due to the presence of the determined possible interferents. The measurement can comprise a spectrum obtained from a material sample, and the spectrum can be an infrared spectrum such as a near infrared spectrum and/or a mid infrared spectrum. The measurement can also comprise a spectrum obtained from a material sample non-invasively. The material sample can include at least one of the following: blood, a component of blood, interstitial fluid, or urine.

One embodiment comprises a method of spectroscopically identifying an interferent in a material sample. The method comprises forming a statistical model of interferent-free spectra, analyzing combinations of material sample spectra and interferent spectra corresponding to varying concentrations of the interferent, and identifying the interferent as a possible interferent if any of the combinations are within predetermined bounds. Identifying the interferent can include determining a Mahalanobis distance between the combinations of material sample spectra and interferent spectra corresponding to varying concentrations of the interferent and the statistical model of interferent-free spectra. Identifying the interferent can further include determining whether the minimum Mahalanobis distance as a function of interferent concentration is sufficiently small relative to the quantiles of a χ² random variable with N degrees of freedom, where N is the number of wavelengths of the spectra.

One embodiment comprises a carrier medium carrying one or more computer readable code segments to instruct a processor to implement a method of spectroscopically identifying an interferent in a material sample. The method comprises forming a statistical model of interferent-free spectra; analyzing combinations of material sample spectra and interferent spectra corresponding to varying concentrations of the interferent; and identifying the interferent as a possible interferent if any of the combinations are within predetermined bounds. Identifying the interferent can include determining a Mahalanobis distance between the combinations of material sample spectra and interferent spectra corresponding to varying concentrations of the interferent and the statistical model of interferent-free spectra. Identifying the interferent can further include determining whether the minimum Mahalanobis distance as a function of interferent concentration is sufficiently small relative to the quantiles of a χ² random variable with N degrees of freedom, where N is the number of wavelengths of the spectra.

One embodiment comprises a method for estimating the concentration of an analyte in a sample from a measurement of the sample. The method comprises identifying, based on the measurement, one or more possible interferents to the measurement of the analyte in the sample; calculating a calibration which reduces error attributable to the one or more possible interferents; applying the calibration to the measurement; and estimating, based on the calibrated measurement, the analyte concentration in the sample. The measurement can comprise a spectrum obtained from a material sample, and the spectrum can be an infrared spectrum such as a near infrared spectrum and/or a mid infrared spectrum. The measurement can also comprise a spectrum obtained from a material sample non-invasively. The material sample can include at least one of the following: blood, a component of blood, interstitial fluid, or urine. The analyte can comprise glucose.

One embodiment comprises a carrier medium carrying one or more computer readable code segments to instruct a processor to implement a method for estimating the concentration of an analyte in a sample from a measurement of the sample. The method comprises identifying, based on the measurement, one or more possible interferents to the measurement of the analyte in the sample; calculating a calibration which reduces error attributable to the one or more possible interferents; applying the calibration to the measurement; and estimating, based on the calibrated measurement, the analyte concentration in the sample. The measurement can comprise a spectrum obtained from a material sample, and the spectrum can be an infrared spectrum such as a near infrared spectrum and/or a mid infrared spectrum. The measurement can also comprise a spectrum obtained from a material sample non-invasively. The material sample can include at least one of the following: blood, a component of blood, interstitial fluid, or urine. The analyte can comprise glucose.

One embodiment comprises a method of generating an average calibration vector for estimating the amount of an analyte from the spectrum of a sample having one or more identified interferents. The method comprises forming a plurality of spectra each including a combination of (i) one of a plurality of interferent-free spectra, each such spectrum having an associated known analyte concentration, and (ii) a spectrum derived from random combinations of possible amounts of the one or more interferents. The method further comprises forming a plurality of first subsets of spectra each including a random selection of the plurality of spectra and defining a corresponding second subset of spectra of the plurality of spectra not included in the first subset. The method further comprises, for each first subset of spectra: (a) generating a calibration vector using the known analyte concentration corresponding to each spectrum; (b) estimating the amount of analyte from each spectrum of the corresponding second subset using the generated calibration vector, and (c) determining a subset-average error between the estimated amount of analyte and the known amount of analyte. The method further comprises calculating an average calibration vector from the calibration vector and determined average error from each subset of spectra to minimize the variance of the error obtained by the use of the averaged calibration. In practicing this method, the sample can comprise a material sample, such as blood, plasma, blood component(s), interstitial fluid, or urine. The spectrum of the sample can be obtained non-invasively. The spectrum of the sample can be an infrared spectrum, a mid infrared spectrum, and/or a near infrared spectrum. In one embodiment, the calibration vector is not required to be perpendicular to the spectra of the determined possible interferents. The calibration vector can minimize errors in the calibration due to the presence of the determined possible interferents.

One embodiment comprises a carrier medium carrying one or more computer readable code segments to instruct a processor to implement a method of generating an average calibration vector for estimating the amount of an analyte from the spectrum of a sample having one or more identified interferents. The method comprises forming a plurality of spectra each including a combination of (i) one of a plurality of interferent-free spectra, each such spectrum having an associated known analyte concentration, and (ii) a spectrum derived from random combinations of possible amounts of the one or more interferents. The method further comprises forming a plurality of first subsets of spectra each including a random selection of the plurality of spectra and defining a corresponding second subset of spectra of the plurality of spectra not included in the first subset. The method further comprises, for each first subset of spectra: (a) generating a calibration vector using the known analyte concentration corresponding to each spectrum; (b) estimating the amount of analyte from each spectrum of the corresponding second subset using the generated calibration vector, and (c) determining a subset-average error between the estimated amount of analyte and the known amount of analyte. The method further comprises calculating an average calibration vector from the calibration vector and determined average error from each subset of spectra to minimize the variance of the error obtained by the use of the averaged calibration. In practicing this method, the sample can comprise a material sample, such as blood, plasma, blood component(s), interstitial fluid, or urine. The spectrum of the sample can be obtained non-invasively. The spectrum of the sample can be an infrared spectrum, a mid infrared spectrum, and/or a near infrared spectrum. In one embodiment, the calibration vector is not required to be perpendicular to the spectra of the determined possible interferents. The calibration vector can minimize errors in the calibration due to the presence of the determined possible interferents.

One embodiment comprises an apparatus for estimating the concentration of an analyte in a sample from a measurement, where the sample may include one or more interferents that affect the measurement. The apparatus comprises means for determining the presence in the sample of possible interferents to the measurement, and means for determining a calibration that reduces errors in the measurement due to the presence of the determined possible interferents. The apparatus can further comprise means for applying the calibration to the measurement, and means for estimating the analyte concentration based on the calibrated measurement. The measurement can be from a person, wherein the means for determining the presence of possible interferents and the means for determining a calibration both include means for comparing the measurement with population measurements, and where the determining does not require the population to include the person. The measurement can comprise a spectrum obtained from a material sample, and the spectrum can be an infrared spectrum, a near infrared spectrum, and/or a mid infrared spectrum. The measurement can also comprise a spectrum obtained from a material sample non-invasively. The material sample can include at least one of the following: blood, plasma or other component(s) of blood, interstitial fluid, or urine. The calibration can be a vector that is not required to be perpendicular to the spectra of the determined possible interferents. The means for determining a calibration can minimize errors in the calibration due to the presence of the determined possible interferents.

One embodiment comprises an apparatus for estimating the concentration of an analyte in a sample from a measurement of the sample. The apparatus comprises means for identifying, based on the measurement, one or more possible interferents to the measurement of the analyte in the sample; means for calculating a calibration which reduces error attributable to the one or more possible interferents; means for applying the calibration to the measurement; and means for estimating, based on the calibrated measurement, the analyte concentration in the sample. The measurement can comprise a spectrum obtained from a material sample, and the spectrum can be an infrared spectrum, a near infrared spectrum, and/or a mid infrared spectrum. The measurement can also comprise a spectrum obtained from a material sample non-invasively. The material sample can include at least one of the following: blood, plasma or other component(s) of blood, interstitial fluid, or urine. The analyte can comprise glucose.

One embodiment comprises an analyte detection system. The system comprises a sensor configured to provide information relating to a measurement of an analyte in a sample; a processor; and stored program instructions. The stored program instructions are executable by the processor such that the system: (a) identifies, based on the measurement, one or more possible interferents to the measurement of the analyte in the sample; (b) calculates a calibration which reduces error attributable to the one or more possible interferents; (c) applies the calibration to the measurement; and (d) estimates, based on the calibrated measurement, the analyte concentration in the sample.

One embodiment comprises an analyte detection system. The system comprises a sensor configured to collect information useful for making a measurement of an analyte in a sample; a processor; and software. The software is executable by the processor such that the system determines the presence in the sample of possible interferents to the measurement; and determines a calibration that reduces errors in the measurement due to the presence of the determined possible interferents.

One embodiment comprises an apparatus for analyzing a material sample. The apparatus comprises an analyte detection system; and a sample element configured for operative engagement with the analyte detection system. The sample element comprises a sample chamber having an internal volume for containing a material sample. The analyte detection system includes a processor and stored program instructions. The program instructions are executable by the processor such that, when the material sample is positioned in the sample chamber and the sample element is operatively engaged with the analyte detection system, the system computes estimated concentrations of the analyte in the material sample in the presence of possible interferents to the estimation of the analyte concentration by determining the presence of possible interferents to the estimation of the analyte concentration and determining a calibration that reduces errors in the estimation due to the presence of the determined possible interferents.

One embodiment comprises a method for estimating a concentration of an analyte in a sample from a measurement of the sample. The method comprises determining, based on the measurement, a list of one or more possible interferents to the measurement of the analyte in the sample. The method further comprises calculating, for each one of the possible interferents on the list, a single interferent analyte concentration based on the presumed presence in the sample of the one interferent and no other interferents from the list. The method also comprises determining an estimated analyte concentration based at least in part on the single interferent analyte concentrations.

One embodiment comprises a carrier medium carrying one or more computer readable code segments to instruct a processor to implement a method for estimating the amount of an analyte in a sample from a measurement of the sample. The method comprises determining, based on the measurement, a list of one or more possible interferents to the measurement of the analyte in the sample. The method further comprises calculating, for each one of the possible interferents on the list, a single interferent analyte concentration based on the presumed presence in the sample of the one interferent and no other interferents from the list. The method also comprises determining an estimated analyte concentration based at least in part on the single interferent analyte concentrations.

One embodiment comprises an apparatus for estimating a concentration of an analyte in a sample from a measurement of the sample. The apparatus comprises means for determining, based on the measurement, a list of one or more possible interferents to the measurement of the analyte in the sample; means for calculating, for each one of the interferents on the list, a single interferent analyte concentration based on the presumed presence in the sample of the one interferent and no other interferents from the list; and means for determining an estimated analyte concentration based at least in part on the single interferent analyte concentrations.

One embodiment comprises an analyte detection system comprising a sensor system and a processor system. The sensor system is configured to provide information relating to a measurement of an analyte in a sample. The processor system is configured to execute stored program instructions such that the analyte detection system determines, based on the measurement, a list of one or more possible interferents to the measurement of the analyte in the sample; calculates, for each one of the interferents on the list, a single interferent analyte concentration based on the presumed presence in the sample of the one interferent and no other interferents from the list; and determines an estimated analyte concentration based at least in part on the single interferent analyte concentrations.

Certain embodiments are summarized above. However, despite the foregoing discussion of certain embodiments, only the appended claims (and not the present summary) are intended to define the invention(s). The summarized embodiments, and other embodiments, will become readily apparent to those skilled in the art from the following detailed description of the preferred embodiments having reference to the attached figures, the invention(s) not being limited to any particular embodiment(s) disclosed.

### BRIEF DESCRIPTION OF THE DRAWINGS

FIG. 1 is a graph illustrating example absorption spectra of various components that may be present in a blood sample;

FIG. 2 is a graph illustrating the change in the example absorption spectra of blood having the indicated additional components of FIG. 1 relative to a Sample Population blood and glucose concentration, where the contribution due to water has been numerically subtracted from the spectra;

FIG. 3 is a block diagram schematically illustrating one embodiment of an analyte measurement system;

FIG. 4 is a flow chart illustrating a first embodiment of an analysis method for determining the concentration of an analyte in the presence of possible interferents;

FIG. 5 is a flow chart illustrating one embodiment of a method for identifying interferents in a sample, which may be used with the first embodiment of FIG. 4;

FIG. 6A is a graph illustrating one embodiment of the method of FIG. 5, and FIG. 6B is a graph further illustrating an embodiment of the method of FIG. 5;

FIG. 7 is a flow chart illustrating one embodiment of a method for generating a model for identifying possible interferents in a sample, which may be used with the first embodiment of FIG. 4;

FIG. 8 is a schematic diagram illustrating one embodiment of a method for generating randomly-scaled interferent spectra;

FIG. 9 is a graph schematically illustrating one embodiment of a distribution of interferent concentrations, which may be used with the embodiment of FIG. 8;

FIG. 10 is a schematic diagram illustrating one embodiment of a method for generating combination interferent spectra;

FIG. 11 is a schematic diagram illustrating one embodiment of a method for generating an interferent-enhanced spectral database;

FIG. 12 is a graph illustrating an example of the effect of interferents on the error of glucose estimation;

FIGS. 13A, 13B, 13C, and 13D each are a graph showing a comparison of an example absorption spectrum of glucose with different interferents taken using two different techniques: a Fourier Transform Infrared (FTIR) spectrometer having an interpolated resolution of 1 cm⁻¹ (solid lines with triangles); and by 25 finite-bandwidth IR filters having a Gaussian profile and full-width half-maximum (FWHM) bandwidth of 28 cm⁻¹ corresponding to a bandwidth that varies from 140 nm at 7.08 μm, up to 279 nm at 10 μm (dashed lines with circles). FIGS. 13A-13D show a comparison of glucose with mannitol (FIG. 13A), dextran (FIG. 13B), n-acetyl L cysteine (FIG. 13C), and procainamide (FIG. 13D), at a concentration level of 1 mg/dL and path length of 1 μm;

FIG. 14 shows a graph of example blood plasma spectra in arbitrary units for 6 blood samples taken from three donors, for a wavelength range from 7 μm to 10 μm, where the symbols on the curves indicate the central wavelengths of the 25 filters;

FIGS. 15A, 15B, 15C, and 15D are graphs of example spectra of the Sample Population of 6 samples having random amounts of mannitol (FIG. 15A), dextran (FIG. 15B), n-acetyl L cysteine (FIG. 15C), and procainamide (FIG. 15D), at concentration levels of 1 mg/dL and path lengths of 1 μm;

FIGS. 16A-16D are graphs comparing example calibration vectors obtained by training in the presence of an interferent, to example calibration vectors obtained by training on clean plasma spectra for mannitol (FIG. 16A), dextran (FIG. 16B), n-acetyl L cysteine (FIG. 16C), and procainamide (FIG. 16D) for water-free spectra;

FIG. 17 schematically illustrates an embodiment of a fluid handling system;

FIG. 18 is a schematic diagram of a first embodiment of a sampling apparatus;

FIG. 19 is a schematic diagram illustrating another embodiment of a sampling apparatus;

FIG. 20 is a graph showing example estimated versus measured glucose values for 4537 spectral samples, with the estimated values determined using an embodiment of an MP1IF (maximum probability IF rejection) technique;

FIG. 21 is a graph showing example estimated versus measured glucose values for 4537 spectral samples, with the estimated values determined using an embodiment of an LW1IF (likelihood-weighted IF rejection) technique; and

FIG. 22 includes two graphs illustrating quantitative differences in scatter between embodiments of the MP1IF technique and the LW1IF technique shown in FIGS. 20 and 21. The upper panel in FIG. 22 illustrates probability density functions, and the lower panel illustrates cumulative probability functions corresponding to the probability density functions in the upper panel. The lower panel also includes a table that lists percentiles for absolute error.

Reference symbols are used in the figures to indicate certain components, aspects or features shown therein, with reference symbols common to more than one figure indicating like components, aspects or features shown therein.

### DETAILED DESCRIPTION OF PREFERRED EMBODIMENTS

Although certain embodiments and examples are disclosed below, it will be understood by those skilled in the art that the inventions disclosed herein extend beyond the specifically disclosed embodiments to other alternative embodiments and/or uses of the inventions and obvious modifications and equivalents thereof. Thus it is intended that the scope of the inventions herein disclosed should not be limited by the particular disclosed embodiments described below. In any method or process disclosed herein, the acts or operations making up the method/process may be performed in any suitable sequence, and are not necessarily limited to any particular disclosed sequence. For purposes of contrasting various embodiments with the prior art, certain aspects and advantages of these embodiments are described where appropriate herein. Of course, it is to be understood that not necessarily all such aspects or advantages may be achieved in accordance with any particular embodiment. Thus, for example, it should be recognized that the various embodiments may be carried out in a manner that achieves or optimizes one advantage or group of advantages as taught herein without necessarily achieving other aspects or advantages as may be taught or suggested herein. While the systems and methods discussed herein may be used for invasive techniques, the systems and methods may also be used for non-invasive techniques or other suitable techniques and may be used in hospitals, healthcare facilities, intensive care units (ICUs), residences, etc.

Several disclosed embodiments are systems and methods for analyzing material sample measurements and for quantifying one or more analytes in the presence of interferents. Interferents can comprise components of a material sample being analyzed for an analyte, where the presence of the interferent affects the quantification of the analyte. Thus, for example, in the spectroscopic analysis of a sample to determine an analyte concentration, an interferent could be a compound having spectroscopic features that overlap with those of the analyte. The presence of such an interferent can introduce errors in the quantification of the analyte. More specifically, the presence of interferents can affect the sensitivity of a measurement technique to the concentration of analytes of interest in a material sample, especially when the system is calibrated in the absence of, or with an unknown amount of, the interferent.

Independently of or in combination with the attributes of interferents described above, interferents can be classified as being endogenous (e.g., originating within the body) or exogenous (e.g., introduced from or produced outside the body). As an example of these classes of interferents, consider the analysis of a blood sample (or a blood component sample or a blood plasma sample) for the analyte glucose. Endogenous interferents include those blood components having origins within the body that affect the quantification of glucose, and may include water, hemoglobin, blood cells, and any other component that naturally occurs in blood. Exogenous interferents include those blood components having origins outside of the body that affect the quantification of glucose, and can include items administered to a person, such as medicaments, drugs, foods or herbs, whether administered orally, intravenously, topically, etc.

Independently of or in combination with the attributes of interferents described above, interferents can comprise components which are possibly, but not necessarily, present in the sample type under analysis. In the example of analyzing samples of blood or blood plasma drawn from patients who are receiving medical treatment, a medicament such as acetaminophen is possibly, but not necessarily, present in this sample type. In contrast, water is necessarily present in such blood or plasma samples.

As used herein, the term “material sample” (or, alternatively, “sample”) is a broad term and is used in its ordinary sense and includes, without limitation, any material which is suitable for analysis. For example, a material sample may comprise whole blood, blood components (e.g., plasma or serum), interstitial fluid, intercellular fluid, saliva, urine, sweat and/or other organic or inorganic materials, or derivatives of any of these materials. As a further example, a material sample comprises any of the above samples as sensed non-invasively in the body. For example, absorption, emission, or other type of optical spectral measurements, which may be combined with acoustical measurements, such as obtained using photoacoustic techniques, may be obtained on a finger, ear, eye, or some other body part.

As used herein, the term “analyte” is a broad term and is used in its ordinary sense and includes, without limitation, any chemical species the presence, concentration, or other property of which is sought in the material sample by an analyte detection system. For example, the analyte(s) include, but not are limited to, glucose, ethanol, insulin, water, carbon dioxide, blood oxygen, cholesterol, bilirubin, ketones, fatty acids, lipoproteins, albumin, urea, creatinine, white blood cells, red blood cells, hemoglobin, oxygenated hemoglobin, carboxyhemoglobin, organic molecules, inorganic molecules, pharmaceuticals, cytochrome, various proteins and chromophores, microcalcifications, electrolytes, sodium, potassium, chloride, bicarbonate, and hormones. As used herein, the term “measurement” is a broad term and is used in its ordinary sense and includes, without limitation, one or more optical, physical, chemical, electrochemical, acoustic, or photoacoustic measurements.

To facilitate an understanding of the inventions, embodiments are discussed herein where one or more analyte concentrations are obtained using spectroscopic measurements of a sample at wavelengths including one or more wavelengths that are identified with the analyte(s). The embodiments disclosed herein are intended as illustrative examples and are not intended to limit, except as claimed, the scope of certain disclosed inventions which are directed to the analysis of measurements in general.

As an example, certain disclosed methods are used to quantitatively estimate the concentration of one specific compound (an analyte) in a mixture from a measurement, where the mixture contains compounds (interferents) that affect the measurement. Certain disclosed embodiments are particularly effective if each analyte and interferent component has a characteristic signature in the measurement, and if the measurement is approximately affine (e.g., includes a linear component and an offset) with respect to the concentration of each analyte and interferent. In one embodiment, a method includes a calibration process including an algorithm for estimating a set of coefficients and one or more offset values that permits the quantitative estimation of an analyte.

In another embodiment, there is provided a method for modifying hybrid linear algorithm (HLA) methods to accommodate a random set of interferents, while retaining a high degree of sensitivity to the desired component. The data used to accommodate the random set of interferents include (a) the signatures of each of the members of the family of potential additional components and (b) the typical quantitative level at which each additional component, if present, is likely to appear. The calibration coefficient is calculated in some embodiments using a hybrid linear analysis (HLA) technique. In certain embodiments, the HLA technique includes constructing a set of spectra that are free of the desired analyte, projecting the analyte's spectrum orthogonally away from the space spanned by the analyte-free calibration spectra, and normalizing the result to produce a unit response. Further description of embodiments of HLA techniques may be found in, for example, “Measurement of Analytes in Human Serum and Whole Blood Samples by Near-Infrared Raman Spectroscopy,” Chapter 4, Andrew J. Berger, Ph. D. thesis, Massachusetts Institute of Technology, 1998, and “An Enhanced Algorithm for Linear Multivariate Calibration,” by Andrew J. Berger, et al., Analytical Chemistry, Vol. 70, No. 3, Feb. 1, 1998, pp. 623-627, the entirety of each of which is hereby incorporated by reference herein. A skilled artisan will recognize that in other embodiments the calibration coefficients may be calculated using other techniques including, for example, regression, partial least squares, and/or principal component analysis.

Thus various alternative embodiments include, but are not limited to, the determination of the presence or concentration of analytes, samples, or interferents other than those disclosed herein. The various alternative embodiments may include other spectroscopic measurements, such as Raman scattering, near infrared spectroscopic methods, and mid infrared spectroscopic methods; non-spectroscopic measurements, such as electrochemical measurement or acoustic measurement; or combinations of different types of measurements. The various alternative embodiments may also include measurements of samples that are chemically and/or physically altered to change the concentration of one or more analytes or interferents and may include measurements on calibrating mixtures.

### Fluid Sampling/Handling and Analyte Detection Systems

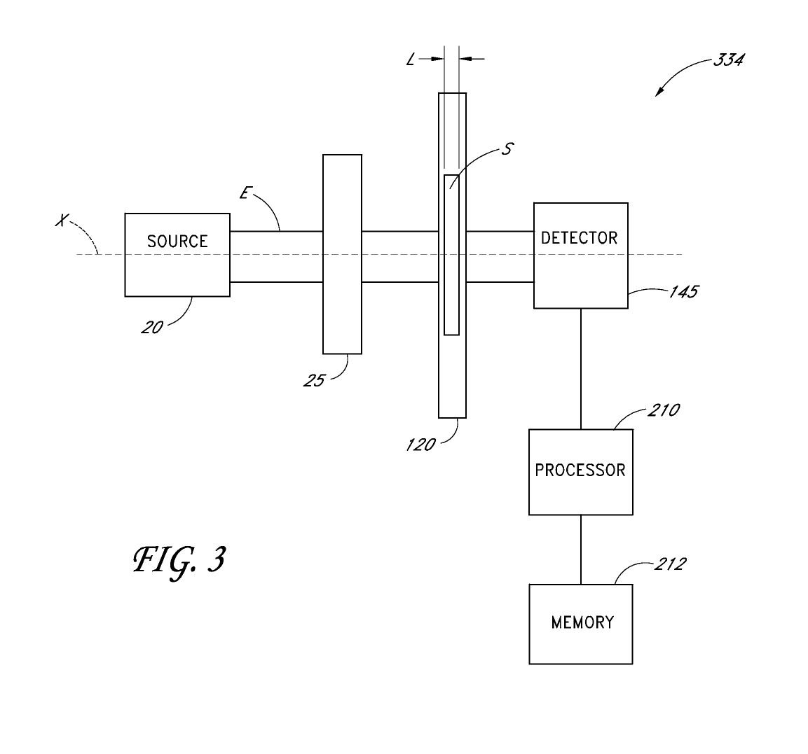

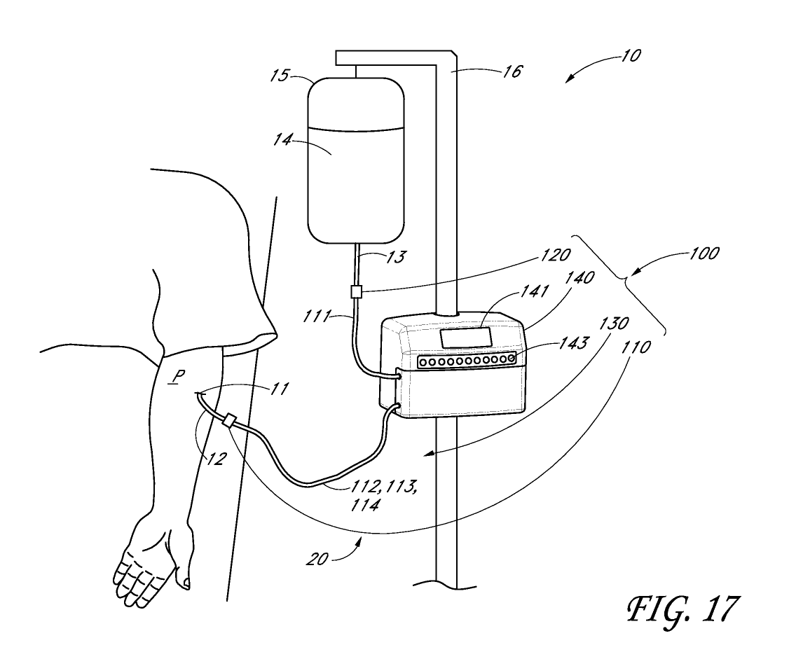

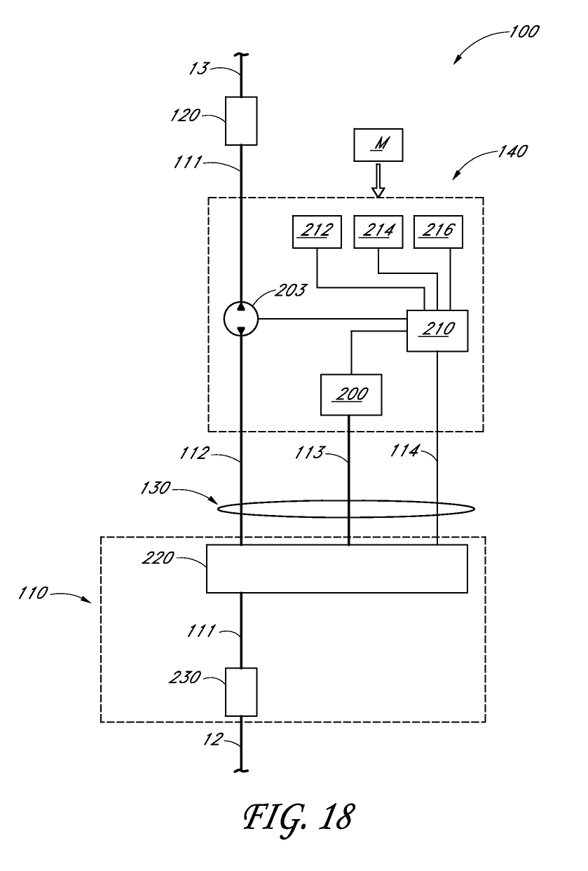

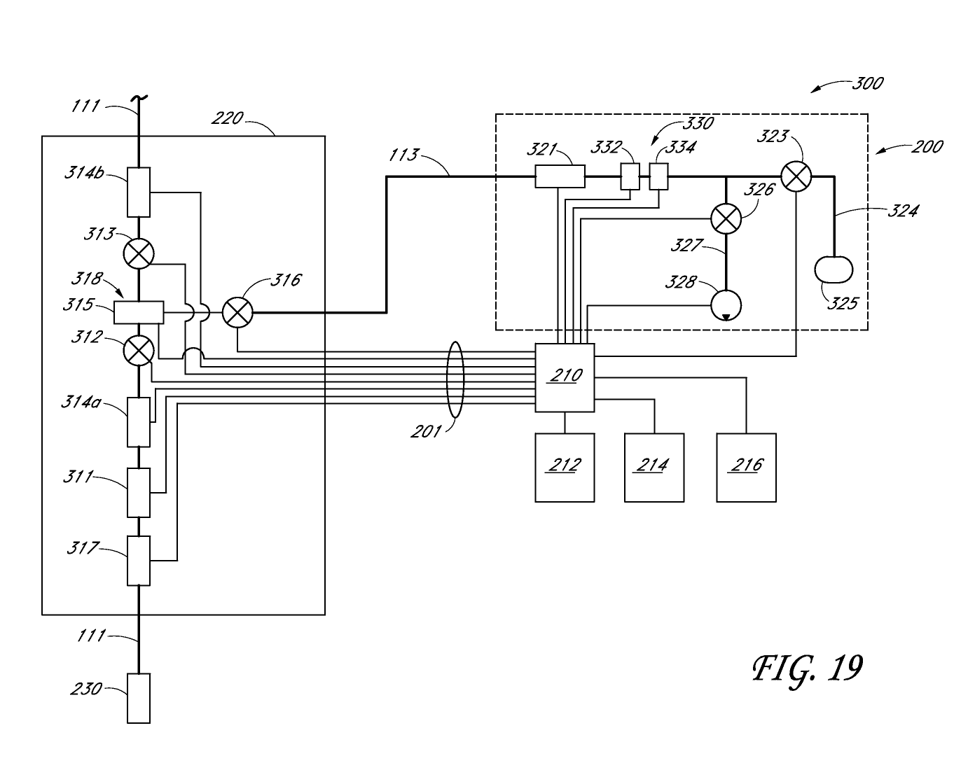

Certain methods, systems, and devices disclosed herein are directed to the determination of the concentration of one or more analytes from measurements of a material sample that may include one or more interferents. As an illustrative example of such measurements, a system for obtaining optical absorption measurements of blood or plasma samples is discussed with reference to FIGS. 3, 17, 18, and 19. FIG. 3 depicts one embodiment of an analyte detection system; FIG. 17 is a schematic diagram of an embodiment of a fluid handling system that can be used to provide material samples to an analyte detection system; FIG. 18 is a schematic diagram of a first embodiment of a sampling apparatus; and FIG. 19 is a schematic diagram showing another embodiment of a sampling apparatus.

FIG. 17 is a schematic diagram of one embodiment of a fluid handling system 10. Fluid handling system 10 includes a container 15 supported by a stand 16 and having an interior that is fillable with a fluid 14, a catheter 11, and a sampling system 100. Fluid handling system 10 includes one or more passageways 20 that form conduits between the container, the sampling system, and the catheter. Generally, sampling system 100 is adapted to accept a fluid supply, such as fluid 14, and to be connected to a patient, including, but not limited to catheter 11 which is used to catheterize a patient P. Fluid 14 includes, but is not limited to, fluids for infusing a patient such as saline, lactated Ringer's solution, or water. Sampling system 100, when so connected, is then capable of providing fluid to the patient. In addition, sampling system 100 is also capable of drawing samples, such as blood, from the patient through catheter 11 and passageways 20, and analyzing at least a portion of the drawn sample. Sampling system 100 measures characteristics of the drawn sample including, but not limited to, one or more of the blood plasma glucose, blood urea nitrogen (BUN), hematocrit, hemoglobin, or lactate levels. Optionally, sampling system 100 includes other devices or sensors to measure other patient or apparatus related information including, but not limited to, patient blood pressure, pressure changes within the sampling system, or sample draw rate. The sampling system 100 may include a user interface including a display 141 that outputs information related to the patient, the fluid sampling process, and/or the fluid handling process. In some embodiments, the display 141 is a touchscreen display that permits user input to the system 100.

In some embodiments, sampling system 100 includes or is in communication with processors that execute or can be instructed to perform certain methods disclosed herein. Thus, for example, one embodiment of sampling system 100 includes one or more processors (not shown) that are programmed or that are provided with programs to analyze device or sensor measurements to determine analyte measurements from a blood sample from patient P. The one or more processors may include a general and/or special purpose computer system. In some embodiments, the processors include one or more floating point gate arrays (FPGAs), programmable logic devices (PLDs), application specific integrated circuits (ASICs), and/or any other suitable processing component. The sampling system 100 may include one or more data storage units including, for example, magnetic storage (e.g., a hard disk drives), optical storage (e.g., optical disks such as CD or DVD storage), and/or semiconductor storage (e.g., flash memory). In certain embodiments, some or all of the processing and/or the storage may be performed at a physically remote location from the system 100. In certain such embodiments, the system 100 may communicate with remote devices over a data network such as, for example, a wide-area network, a local-area network, a hospital information system (HIS), the Internet, the World-Wide-Web, and so forth. The communication may be via wired and/or wireless techniques.

More specifically, FIG. 17 shows sampling system 100 as including a patient connector 110, a fluid handling and analysis apparatus 140, and a connector 120. Sampling system 100 may include combinations of passageways, fluid control and measurement devices, and analysis devices to direct, sample, and analyze fluid. Passageways 20 of sampling system 100 include a first passageway 111 from connector 120 to fluid handling and analysis apparatus 140, a second passageway 112 from the fluid handling and analysis apparatus to patient connector 110, and a third passageway 113 from the patient connector to the fluid handling and analysis apparatus. The reference of passageways 20 as including one or more passageway, for example passageways 111, 112, and 113 are provided to facilitate discussion of the system. It is understood that passageways may include one or more separate components and may include other intervening components including, but not limited to, pumps, valves, manifolds, and analytic equipment.

As used herein, the term “passageway” is a broad term and is used in its ordinary sense and includes, without limitation except as explicitly stated, as any opening through a material through which a fluid may pass so as to act as a conduit. Passageways include, but are not limited to, flexible, inflexible or partially flexible tubes, laminated structures having openings, bores through materials, or any other structure that can act as a conduit and any combination or connections thereof. The internal surfaces of passageways that provide fluid to a patient or that are used to transport blood are preferably biocompatible materials, including but not limited to silicone, polyetheretherketone (PEEK), or polyethylene (PE). One type of preferred passageway is a flexible tube having a fluid contacting surface formed from a biocompatible material. A passageway, as used herein, also includes separable portions that, when connected, form a passageway.

The inner passageway surfaces may include coatings of various sorts to enhance certain properties of the conduit, such as coatings that affect the ability of blood to clot or to reduce friction resulting from fluid flow. Coatings include, but are not limited to, molecular or ionic treatments.

As used herein, the term “connector” is a broad term and is used in its ordinary sense and includes, without limitation except as explicitly stated, as a device that connects passageways or electrical wires to provide communication on either side of the connector. Some connectors contemplated herein include a device for connecting any opening through which a fluid may pass. In some embodiments, a connector may also house devices for the measurement, control, and preparation of fluid, as described in several of the embodiments.

Fluid handling and analysis apparatus 140 may control the flow of fluids through passageways 20 and the analysis of samples drawn from a patient P. as described subsequently. Fluid handling and analysis apparatus 140 includes a display 141 and input devices, such as buttons 143. The display 141 may provide information on the operation or results of an analysis performed by fluid handling and analysis apparatus 140. In one embodiment, the display 141 indicates the function of buttons 143, which are used to input information into fluid handling and analysis apparatus 140. Information that may be input into or obtained by fluid handling and analysis apparatus 140 includes, but is not limited to, a required infusion or dosage rate, sampling rate, or patient specific information which may include, but is not limited to, a patient identification number or medical information. In another embodiment, fluid handling and analysis apparatus 140 obtains information on patient P over a communications network, for example an hospital communication network having patient specific information which may include, but is not limited to, medical conditions, medications being administered, laboratory blood reports, gender, and weight. As one example of the use of fluid handling system 10, FIG. 17 shows catheter 11 connected to patient P.

As discussed subsequently, fluid handling system 10 may catheterize a patient's vein or artery. Sampling system 100 is releasably connectable to container 15 and catheter 11. Thus, for example, FIG. 17 shows container 15 as including a tube 13 to provide for the passage of fluid to, or from, the container, and catheter 11 as including a tube 12 external to the patient. Connector 120 is adapted to join tube 13 and passageway 111. Patient connector 110 is adapted to join tube 12 and to provide for a connection between passageways 112 and 113.

Patient connector 110 may also include devices that control, direct, process, or otherwise affect the flow through passageways 112 and 113. In some embodiments, one or more control or electrical lines 114 are provided to exchange signals between patient connector 110 and fluid handling and analysis apparatus 140. As shown in FIG. 17, sampling system 100 may also include passageways 112 and 113, and electrical lines 114, when present. The passageways and electrical lines between apparatus 140 and patient connector 110 are referred to, with out limitation, as a bundle 130.

In various embodiments, fluid handling and analysis apparatus 140 and/or patient connector 110, includes other elements (not shown in FIG. 17) that include, but are not limited to: fluid control elements, including but not limited to valves and pumps; fluid sensors, including but not limited to pressure sensors, temperature sensors, hematocrit sensors, hemoglobin sensors, calorimetric sensors, and gas (or “bubble”) sensors; fluid conditioning elements, including but not limited to gas injectors, gas filters, and blood plasma separators; and wireless communication devices to permit the transfer of information within the sampling system or between sampling system 100 and a wireless network.

In one embodiment, patient connector 110 includes devices to determine when blood has displaced fluid 14 at the connector end, and thus provides an indication of when a sample is available for being drawn through passageway 113 for sampling. The presence of such a device at patient connector 110 allows for the operation of fluid handling system 10 for analyzing samples without regard to the actual length of tube 12. Accordingly, bundle 130 may include elements to provide fluids, including air, or information communication between patient connector 110 and fluid handling and analysis apparatus 140 including, but not limited to, one or more other passageways and/or wires.

In one embodiment of sampling system 100, the passageways and lines of bundle 130 are sufficiently long to permit locating patient connector 110 near patient P, for example with tube 12 having a length of less than 0.1 to 0.5 meters, or preferably approximately 0.15 meters and with fluid handling and analysis apparatus 140 located at a convenient distance, for example on a nearby stand 16. Thus, for example, bundle 130 is from 0.3 to 3 meters, or more preferably from 1.5 to 2.0 meters in length. It is preferred, though not required, that patient connector 110 and connector 120 include removable connectors adapted for fitting to tubes 12 and 13, respectively. Thus, in one embodiment, container 15/tube 13 and catheter 11/tube 12 are both standard medical components, and sampling system 100 allows for the easy connection and disconnection of one or both of the container and catheter from fluid handling system 10.

In another embodiment of sampling system 100, tubes 12 and 13 and a substantial portion of passageways 111 and 112 have approximately the same internal cross-sectional area. It is preferred, though not required, that the internal cross-sectional area of passageway 113 is less than that of passageways 111 and 112. As described subsequently, the difference in areas permits fluid handling system 10 to transfer a small sample volume of blood from patient connector 110 into fluid handling and analysis apparatus 140.

Thus, for example, in one embodiment passageways 111 and 112 are formed from a tube having an inner diameter from 0.3 millimeter to 1.50 millimeter, or more preferably having a diameter from 0.60 millimeter to 1.2 millimeter. Passageway 113 is formed from a tube having an inner diameter from 0.3 millimeter to 1.5 millimeter, or more preferably having an inner diameter of from 0.6 millimeter to 1.2 millimeter.

While FIG. 17 shows sampling system 100 connecting a patient to a fluid source, the scope of the present disclosure is not meant to be limited to this embodiment. Alternative embodiments include, but are not limited to, a greater or fewer number of connectors or passageways, or the connectors may be located at different locations within fluid handling system 10, and alternate fluid paths. Thus, for example, passageways 111 and 112 may be formed from one tube, or may be formed from two or more coupled tubes including, for example, branches to other tubes within sampling system 100, and/or there may be additional branches for infusing or obtaining samples from a patient. In addition, patient connector 110 and connector 120 and sampling system 100 alternatively include additional pumps and/or valves to control the flow of fluid as described below. In some embodiments, the fluid handling system 10 can be in fluid communication with an extracorporeal fluid conduit containing a volume of a bodily fluid. For example, in lieu of the depicted tube 12, any suitable extracorporeal fluid conduit, such as catheter, IV tube, or an IV network, can be connected to the sampling system 100. The extracorporeal fluid conduit need not be attached to the patient P; for example, the extracorporeal fluid conduit can be in fluid communication with a container of the bodily fluid of interest (e.g., blood), or the extracorporeal fluid conduit can serve as a stand-alone volume of the bodily fluid of interest.

FIG. 18 is a schematic of a sampling system 100 configured to analyze blood from patient P which may be generally similar to the embodiment of the sampling system illustrated in FIG. 17, except as further detailed below. Where possible, similar elements are identified with identical reference numerals in the depiction of the embodiments of FIGS. 17 and 18. FIG. 18 shows patient connector 110 as including a sampling assembly 220 and a connector 230, portions of passageways 111 and 113, and electrical lines 114, and fluid handling and analysis apparatus 140 as including a pump 203, a sampling unit 200, and a controller 210. Passageway 111 provides fluid communication between connector 120 and pump 203 and passageway 113 provides fluid communication between pump 203 and connector 110. As described subsequently in several embodiments, sampling unit 200 may include one or more passageways, pumps and/or valves, and sampling assembly 220 may include passageways, sensors, valves, and/or sample detection devices.

Controller 210 collects information from sensors and devices within sampling assembly 220, from sensors and analytical equipment within sampling unit 200, and provides coordinated signals to control pump 203 and pumps and valves, if present, in sampling assembly 220. Thus, for example, controller 210 is in communication with pump 203, sampling unit 200, and sampling assembly 220 through electrical lines 114.

Controller 210 also has access to memory 212, which may contain some or all of the programming instructions for analyzing measurements from sensors and analytical equipment within sampling unit 200 according to one or more of the methods described herein. Optionally, controller 210 and/or memory 212 has access to a media reader 214 that accepts a media M and/or a communications link 216 to provide programming instructions to accomplish one or more of the methods described herein. Media M includes, but is not limited to, optical media such as a DVD or a CD-ROM and semiconductor media such as flash memory. Communications link 216 includes, but is not limited to, a wired or wireless Internet connection.

In some embodiments, controller 210 contains or is provided with programming instructions through memory 212, media reader 214, and/or communications link 216, to perform any one or combination of the methods described herein, including but not limited to the disclosed methods of measurement analysis, interferent determination, and/or calibration coefficient generation. Additionally or alternatively communications link 216 is used to provide measurements from sampling unit 200 for the performance of one or more of the methods described herein by one or more other processors.

In other embodiments, communications link 216 establishes a connection to a computer containing patient specific information that may be used by certain methods described herein. Thus, for example, information regarding the patient's medical condition or parameters that affect the determination of analyte concentrations may be transferred from a computer containing patient specific information to memory 212 to aid in the analysis. Examples of such patient specific information include, but are not limited to, current and/or past concentrations of analyte(s) and/or interferent(s) as determined by other analytical equipment.

Fluid handling and analysis apparatus 140 includes the ability to pump in a forward direction (towards the patient) and in a reverse direction (away from the patient). Thus, for example, pump 203 may direct fluid 14 into patient P or draw a sample, such as a blood sample from patient P, from catheter 11 to sampling assembly 220, where it is further directed through passageway 113 to sampling unit 200 for analysis. Preferably, pump 203 provides a forward flow rate at least sufficient to keep the patient vascular line open. In one embodiment, the forward flow rate is from 1 to 5 ml/hr. When operated in a reverse direction, fluid handling and analysis apparatus 140 includes the ability to draw a sample from the patient to sampling assembly 220 and through passageway 113. In one embodiment, pump 203 provides a reverse flow to draw blood to sampling assembly 220, preferably by a sufficient distance past the sampling assembly to ensure that the sampling assembly contains an undiluted blood sample. In one embodiment, passageway 113 has an inside diameter of from 25 to 200 microns, or more preferably from 50 to 100 microns. Sampling unit 200 extracts a small sample, for example from 10 to 100 microliters of blood, or more preferably approximately 40 microliters volume of blood, from sampling assembly 220.

In one embodiment, pump 203 is a directionally controllable pump that acts on a flexible portion of passageway 111. Examples of a single, directionally controllable pump include, but are not limited to a reversible peristaltic pump or two unidirectional pumps that work in concert with valves to provide flow in two directions. In an alternative embodiment, pump 203 includes a combination of pumps, including but not limited to displacement pumps, such as a syringe, and/or valve to provide bi-directional flow control through passageway 111.

Controller 210 includes one or more processors for controlling the operation of fluid handling system 10 and for analyzing sample measurements from fluid handling and analysis apparatus 140. Controller 210 also accepts input from buttons 143 and provides information on display 141. Optionally, controller 210 is in bi-directional communication with a wired or wireless communication system, for example a hospital network for patient information. The one or more processors comprising controller 210 may include one or more processors that are located either within fluid handling and analysis apparatus 140 or that are networked to the unit.

The control of fluid handling system 10 by controller 210 may include, but is not limited to, controlling fluid flow to infuse a patient and to sample, prepare, and analyze samples. The analysis of measurements obtained by fluid handling and analysis apparatus 140 of may include, but is not limited to, analyzing samples based on inputted patient specific information, from information obtained from a database regarding patient specific information, or from information provided over a network to controller 210 used in the analysis of measurements by apparatus 140.

Fluid handling system 10 provides for the infusion and sampling of a patient blood as follows. With fluid handling system 10 connected to bag 15 having fluid 14 and to a patient P, controller 210 infuses a patient by operating pump 203 to direct the fluid into the patient. Thus, for example, in one embodiment, the controller directs that samples be obtained from a patient by operating pump 203 to draw a sample. In one embodiment, pump 203 draws a predetermined sample volume, sufficient to provide a sample to sampling assembly 220. In another embodiment, pump 203 draws a sample until a device within sampling assembly 220 indicates that the sample has reached the patient connector 110. As an example, one such indication is provided by a sensor that detects changes in the color of the sample. Another example is the use of a device that indicates changes in the material within passageway 111 including, but not limited to, a decrease in the amount of fluid 14, a change with time in the amount of fluid, a measure of the amount of hemoglobin, or an indication of a change from fluid to blood in the passageway.

When the sample reaches sampling assembly 220, controller 210 provides an operating signal to valves and/or pumps in sampling system 100 (not shown) to draw the sample from sampling assembly 220 into sampling unit 200. After a sample is drawn towards sampling unit 200, controller 210 then provides signals to pump 203 to resume infusing the patient. In one embodiment, controller 210 provides signals to pump 203 to resume infusing the patient while the sample is being drawn from sampling assembly 220. In an alternative embodiment, controller 210 provides signals to pump 203 to stop infusing the patient while the sample is being drawn from sampling assembly 220. In another alternative embodiment, controller 210 provides signals to pump 203 to slow the drawing of blood from the patient while the sample is being drawn from sampling assembly 220.

In another alternative embodiment, controller 210 monitors indications of obstructions in passageways or catheterized blood vessels during reverse pumping and moderates the pumping rate and/or direction of pump 203 accordingly. Thus, for example, obstructed flow from an obstructed or kinked passageway or of a collapsing or collapsed catheterized blood vessel that is being pumped will result in a lower pressure than an unobstructed flow. In one embodiment, obstructions are monitored using a pressure sensor in sampling assembly 220 or along passageways 20. If the pressure begins to decrease during pumping, or reaches a value that is lower than a predetermined value then controller 210 directs pump 203 to decrease the reverse pumping rate, stop pumping, or pump in the forward direction in an effort to reestablish unobstructed pumping.

FIG. 19 is a schematic showing details of a sampling system 300 which may be generally similar to the embodiments of sampling system 100 as illustrated in FIGS. 17 and 18, except as further detailed below. Sampling system 300 includes sampling assembly 220 having, along passageway 112: connector 230 for connecting to tube 12, a pressure sensor 317, a calorimetric sensor 311, a first bubble sensor 314a, a first valve 312, a second valve 313, and a second bubble sensor 314b. Passageway 113 forms a “T” with passageway 111 at a junction 318 that is positioned between the first valve 312 and second valve 313, and includes a gas injector manifold 315 and a third valve 316. Electrical lines 114 comprise control and/or signal lines extending from calorimetric sensor 311, first, second, and third valves (312, 313, 316), first and second bubble sensors (314a, 314b), gas injector 315, and pressure sensor 317. Sampling system 300 also includes sampling unit 200 which has a bubble sensor 321, a sample analysis device 330, a first valve 323a, a waste receptacle 325, a second valve 323b, and a pump 328. Passageway 113 forms a “T” to form a waste line 324 and a pump line 327.

It is preferred, though not necessary, that the sensors of sampling system 100 are adapted to accept a passageway through which a sample may flow and that sense through the walls of the passageway. As described subsequently, this arrangement allows for the sensors to be reusable and for the passageways to be disposable. It is also preferred, though not necessary, that the passageway is smooth and without abrupt dimensional changes which may damage blood or prevent smooth flow of blood. In addition, is also preferred that the passageways that deliver blood from the patient to the analyzer not contain gaps or size changes that permit fluid to stagnate and not be transported through the passageway.

In one embodiment, the respective passageways on which valves 312, 313, 316, and 323 are situated along passageways that are flexible tubes, and valves 312, 313, 316, and 323 are “pinch valves,” in which one or more movable surfaces compress the tube to restrict or stop flow therethrough. In one embodiment, the pinch valves include one or more moving surfaces that are actuated to move together and “pinch” a flexible passageway to stop flow therethrough. Examples of a pinch valve include, for example, Model PV256 Low Power Pinch Valve (Instech Laboratories, Inc., Plymouth Meeting, Pa.). Alternatively, one or more of valves 312, 313, 316, and 323 may be other valves for controlling the flow through their respective passageways.

Colorimetric sensor 311 accepts or forms a portion of passageway 111 and provides an indication of the presence or absence of blood within the passageway. In one embodiment, calorimetric sensor 311 permits controller 210 to differentiate between fluid 14 and blood. Preferably, calorimetric sensor 311 is adapted to receive a tube or other passageway for detecting blood. This permits, for example, a disposable tube to be placed into or through a reusable calorimetric sensor. In an alternative embodiment, calorimetric sensor 311 is located adjacent to bubble sensor 314b. Examples of a calorimetric sensor include, for example, an Optical Blood Leak/Blood vs. Saline Detector available from Introtek International (Edgewood, N.J.).

Sampling system 300 injects a gas—referred to herein and without limitation as a “bubble”—into passageway 113. Specifically, sampling system 300 includes gas injector manifold 315 at or near junction 318 to inject one or more bubbles, each separated by liquid, into passageway 113. The use of bubbles is useful in preventing longitudinal mixing of liquids as they flow through passageways both in the delivery of a sample for analysis with dilution and for cleaning passageways between samples. Thus, for example the fluid in passageway 113 includes, in one embodiment, two volumes of liquids, such as sample S or fluid 14 separated by a bubble, or multiple volumes of liquid each separated by a bubble therebetween.

Bubble sensors 314a, 314b and 321 each accept or form a portion of passageway 112 or 113 and provide an indication of the presence of air, or the change between the flow of a fluid and the flow of air, through the passageway. Examples of bubble sensors include, but are not limited to ultrasonic or optical sensors, that can detect the difference between small bubbles or foam from liquid in the passageway. Once such bubble detector is an MEC Series Air Bubble/Liquid Detection Sensor (Introtek International, Edgewood, N.Y.). Preferably, bubble sensor 314a, 314b, and 321 are each adapted to receive a tube or other passageway for detecting bubbles. This permits, for example, a disposable tube to be placed through a reusable bubble sensor.

Pressure sensor 317 accepts or forms a portion of passageway 111 and provides an indication or measurement of a fluid within the passageway. When all valves between pressure sensor 317 and catheter 11 are open, pressure sensor 317 provides an indication or measurement of the pressure within the patient's catheterized blood vessel. In one embodiment of a method, the output of pressure sensor 317 is provided to controller 210 to regulate the operation of pump 203. Thus, for example, a pressure measured by pressure sensor 317 above a predetermined value is taken as indicative of a properly working system, and a pressure below the predetermined value is taken as indicative of excessive pumping due to, for example, a blocked passageway or blood vessel. Thus, for example, with pump 203 operating to draw blood from patient P, if the pressure as measured by pressure sensor 317 is within a range of normal blood pressures, it may be assumed that blood is being drawn from the patient and pumping continues. However, if the pressure as measured by pressure sensor 317 falls below some level, then controller 210 instructs pump 203 to slow or to be operated in a forward direction to reopen the blood vessel. One such pressure sensor is a Deltran IV part number DPT-412 (Utah Medical Products, Midvale, Utah).

Sample analysis device 330 receives a sample and performs an analysis. In several embodiments, device 330 is configured to prepare the sample for analysis. Thus, for example, device 330 may include a sample preparation unit 332 and an analyte detection system 334, where the sample preparation unit is located between the patient and the analyte detection system. In general, sample preparation occurs between sampling and analysis. Thus, for example, sample preparation unit 332 may take place removed from analyte detection, for example within sampling assembly 220, or may take place adjacent or within analyte detection system 334.

In one embodiment, sample preparation unit 332 removes separates blood plasma from a whole blood sample or removes contaminants from a blood sample and thus comprises one or more devices including, but not limited to, a filter, membrane, centrifuge, or some combination thereof. The preparation of blood plasma permits, for example, an optical measurement to be made with fewer particles, such as blood cells, that might scatter light, and/or provides for the direct determination of analyte concentrations in the plasma. In alternative embodiments, analyte detection system 334 is adapted to analyze the sample directly and sample preparation unit 332 is not required.

### Spectroscopic Analyte Detection Systems

The analyte detection system 334 is particularly suited for detecting the concentration of one or more analytes in a material sample S, by detecting energy transmitted through the sample. With reference to FIG. 3, this embodiment of the analyte detection system 334 comprises an energy source 20 disposed along a major axis X of the system 334. When activated, the energy source 20 generates an energy beam E which advances from the energy source 20 along the major axis X. Energy beam E passes from source 20, through a sample element or cuvette 120, which supports or contains the material sample S, and then reaches a detector 145. The interaction of energy beam E with sample S occurs over a pathlength L along major axis X. Detector 145 responds to radiation incident thereon by generating an electrical signal and passing the signal to a processor 210 for analysis.

Detection system 334 provides for the measurement of sample S according to the wavelength of energy interacting with sample S. In general, this measurement may be accomplished with beam E of varying wavelengths, or optionally by providing a beam E having a broad range of wavelengths and providing filters between source 20 and detector 145 for selecting a narrower wavelength range for measurement. In one embodiment, the energy source 20 comprises an infrared source and the energy beam E comprises an infrared energy beam, and energy beam E passes through a filter 25, also situated on the major axis X. Based on the signal(s) passed to it by the detector 145, the processor computes the concentration of the analyte(s) of interest in the sample S, and/or the absorbance/transmittance characteristics of the sample S at one or more wavelengths or wavelength bands employed to analyze the sample.

In some embodiments, the processor 210 computes the concentration(s), absorbance(s), transmittance(s), etc. by executing a data processing algorithm or program instructions residing within memory 212 accessible by the processor 210. Any one or combination of the methods disclosed herein (including but not limited to the disclosed methods of measurement analysis, interferent determination, and/or calibration coefficient generation) may be provided to memory 212 or processor 210 via communications with a computer network or by receiving computer readable media (not shown). In addition, any one or combination of the methods disclosed herein may be updated, changed, or otherwise modified by providing new or updated programming, data, computer-readable code, etc. to processor 210. The processor 210 may be embodied as one or more microprocessors, general purpose computers, special purpose computers, or a combination thereof. The processor 210 may include processing components located physically remotely from the analyte detection system 334. The methods described herein may be embodied in computer software (e.g., executable instructions) stored on any form of computer-readable media. The computer software may be executable by the processor 210 or any suitable computer system.

In one embodiment of analyte detection system 334, filter 25 comprises a varying-passband filter, to facilitate changing, over time and/or during a measurement taken with the detection system 334, the wavelength or wavelength band of the energy beam E that may pass the filter 25 for use in analyzing the sample S. When the energy beam E is filtered with a varying-passband filter, the absorption/transmittance characteristics of the sample S can be analyzed at a number of wavelengths or wavelength bands in a separate, sequential manner. As an example, assume that it is desired to analyze the sample S at N separate wavelengths (Wavelength 1 through Wavelength N). The varying-passband filter is first operated or tuned to permit the energy beam E to pass at Wavelength 1, while substantially blocking the beam E at most or all other wavelengths to which the detector 145 is sensitive (including Wavelengths 2-N). The absorption/transmittance properties of the sample S are then measured at Wavelength 1, based on the beam E that passes through the sample S and reaches the detector 145. The varying-passband filter is then operated or tuned to permit the energy beam E to pass at Wavelength 2, while substantially blocking other wavelengths as discussed above; the sample S is then analyzed at Wavelength 2 as was done at Wavelength 1. This process is repeated until all of the wavelengths of interest have been employed to analyze the sample S. The collected absorption/transmittance data can then be analyzed by the processor 210 to determine the concentration of the analyte(s) of interest in the material sample S.

The measured spectrum of sample S is referred to herein in general as Cₛ(λᵢ), that is, a wavelength dependent spectrum in which CS is, for example, a transmittance, an absorbance, an optical density, or some other measure of the optical properties of sample S having values computed or measured at or about each of a number of wavelengths λᵢ, where i ranges over the number of measurements taken. The measurement Cₛ(λᵢ) is a linear array of measurements that is alternatively written as Csᵢ.

The spectral region of analyte detection system 334 depends on the analysis technique and the analyte and mixtures of interest. For example, one useful spectral region for the measurement of glucose concentration in blood or blood plasma using absorption spectroscopy is the mid infrared (for example, from about 4 microns to about 11 microns). In an alternative embodiment, glucose concentration is determined using near infrared spectroscopy. In some embodiments, both near infrared and mid infrared spectroscopy may be used.

In one embodiment of system 334, energy source 20 produces a beam E having an output in the range of about 4 microns to about 11 microns. Although water is the main contributor to the total absorption across this spectral region, the peaks and other structures present in the blood spectrum from about 6.8 microns to 10.5 microns are due to the absorption spectra of other blood components. The 4 to 11 micron region has been found advantageous because glucose has a strong absorption peak structure from about 8.5 to 10 microns, whereas most other blood constituents have a low and flat absorption spectrum in the 8.5 to 10 micron range. The main exceptions are water and hemoglobin, both of which are interferents in this region.

The amount of spectral detail provided by system 334 depends on the analysis technique and the analyte and mixture of interest. For example, the measurement of glucose in blood by mid-IR absorption spectroscopy can be accomplished with from 11 to 25 filters within a spectral region. In one embodiment of system 334, energy source 20 produces a beam E having an output in the range of about 4 microns to about 11 microns, and filter 25 include a number of narrow band filters within this range, each allowing only energy of a certain wavelength or wavelength band to pass therethrough. Thus, for example, one embodiment filter 25 includes a filter wheel having 11 filters, each having a nominal wavelength approximately equal to one of the following: 3 μm, 4.06 μm, 4.6 μm, 4.9 μm, 5.25 μm, 6.12 μm, 6.47 μm, 7.98 μm, 8.35 μm, 9.65 μm, and 12.2 μm.

Blood samples may be prepared and analyzed by system 334 in a variety of configurations. In one embodiment, sample S is obtained by drawing blood, either using a syringe or as part of a blood flow system, and transferring the blood into cuvette 120. In another embodiment, sample S is drawn into a sample container that is a cuvette 120 adapted for insertion into system 334. In yet another embodiment, sample S is blood plasma that is separated from whole blood by a filter or centrifuge before being placed in cuvette 120.

### Methods and Systems for Analyte Measurement

This section discusses a number of computational methods or algorithms which may be used to calculate the concentration of the analyte(s) of interest in the sample S, and/or to compute other measures that may be used in support of calculations of analyte concentrations. Any one or combination of the algorithms disclosed in this section may reside as program instructions stored in the memory 212 so as to be accessible for execution by the processor 210 of the analyte detection system 334 to compute the concentration of the analyte(s) of interest in the sample, or other relevant measures.

Certain methods disclosed herein are directed to the estimation of analyte concentrations in a material sample in the possible presence of an interferent. In certain embodiments, any one or combination of the methods disclosed herein may be accessible to and executable by the processor 210 of the system 334. In some embodiments, processors additional to or alternate from the processor 210 are used to perform some or all of the methods. The processor 210 may be connected to a computer network, and data obtained from system 334 can be transmitted over the network to one or more remote computers that implement the methods. The disclosed methods can include the manipulation of data related to sample measurements and other information supplied to the methods (including, but not limited to, interferent spectra, sample population models, and threshold values, as described subsequently). Any or all of this information, as well as specific algorithms, may be updated or changed to improve the method or provide additional information, such as additional analytes or interferents.

Certain disclosed methods generate a “calibration coefficient” that, when multiplied by a measurement, produces an estimate of an analyte concentration. Both the calibration coefficient and the measurement can comprise arrays of numbers. The calibration coefficient may be calculated to minimize or reduce the sensitivity of the calibration to the presence of interferents that are identified as possibly being present in the sample. Certain methods described herein generate a calibration coefficient by: 1) identifying the presence of possible interferents; and 2) using information related to the identified interferents to generate the calibration coefficient. These certain methods do not require that the information related to the interferents includes an estimate of the interferent concentration—they merely require that the interferents be identified as possibly present in a sample. In one embodiment, the method uses a set of training spectra each having known analyte concentration(s) and produces a calibration that minimizes the variation in estimated analyte concentration with interferent concentration. The resulting calibration coefficient is proportional to analyte concentration(s) and, on average, is not sensitive to interferent concentrations.

In one embodiment, it is not required (though not prohibited either) that the training spectra include any spectrum from the individual whose analyte concentration is to be determined. That is, the term “training” when used in reference to the disclosed methods does not require training using measurements from the individual whose analyte concentration will be estimated (e.g., by analyzing a bodily fluid sample drawn from the individual).

Several terms are used herein to describe the estimation process. As used herein, the term “Sample Population” is a broad term and includes, without limitation, a large number of samples having measurements that are used in the computation of a calibration—in other words, used to train the method of generating a calibration. For an embodiment involving the spectroscopic determination of glucose concentration, the Sample Population measurements can each include a spectrum (analysis measurement) and a glucose concentration (analyte measurement). In one embodiment, the Sample Population measurements are stored in a database, referred to herein as a “Population Database.”

The Sample Population may or may not be derived from measurements of material samples that contain interferents to the measurement of the analyte(s) of interest. One distinction made herein between different interferents is based on whether the interferent is present in both the Sample Population and the sample being measured, or only in the sample. As used herein, the term “Type-A interferent” refers to an interferent that is present in both the Sample Population and in the material sample being measured to determine an analyte concentration. In certain methods it is assumed that the Sample Population includes only interferents that are endogenous, and does not include any exogenous interferents, and thus Type-A interferents are endogenous. The number of Type-A interferents depends on the measurement and analyte(s) of interest, and may number, in general, from zero to a very large number. The material sample being measured, for example the sample S, may also include interferents that are not present in the Sample Population. As used herein, the term “Type-B interferent” refers to an interferent that is either: 1) not found in the Sample Population but that is found in the material sample being measured (e.g., an exogenous interferent), or 2) is found naturally in the Sample Population, but is at abnormally high concentrations in the material sample (e.g., an endogenous interferent). Examples of a Type-B exogenous interferent may include medications, and examples of Type-B endogenous interferents may include urea in persons suffering from renal failure. In the example of mid-infrared (mid-IR) spectroscopic absorption measurement of glucose in blood, water is found in all blood samples, and is thus a Type-A interferent. For a Sample Population made up of individuals who are not taking intravenous drugs, and a material sample taken from a hospital patient who is being administered a selected intravenous drug, the selected drug is a Type-B interferent.

In one embodiment, a list of one or more possible Type-B Interferents is referred to herein as forming a “Library of Interferents,” and each interferent in the library is referred to as a “Library Interferent.” The Library Interferents include exogenous interferents and endogenous interferents that may be present in a material sample due, for example, to a medical condition causing abnormally high concentrations of the endogenous interferent.

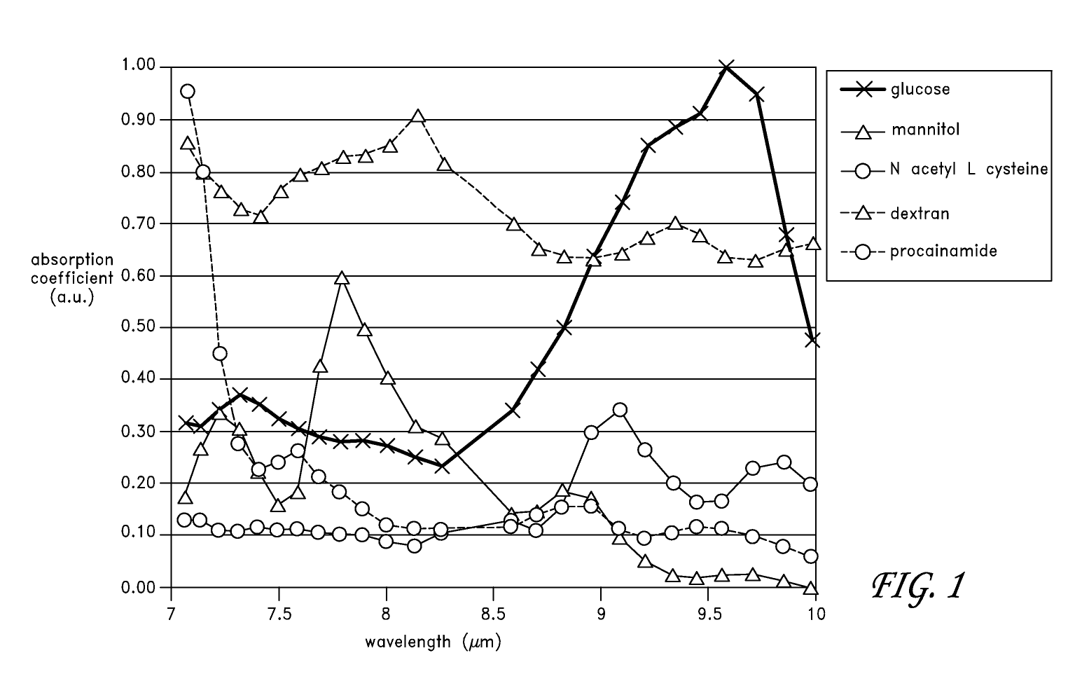

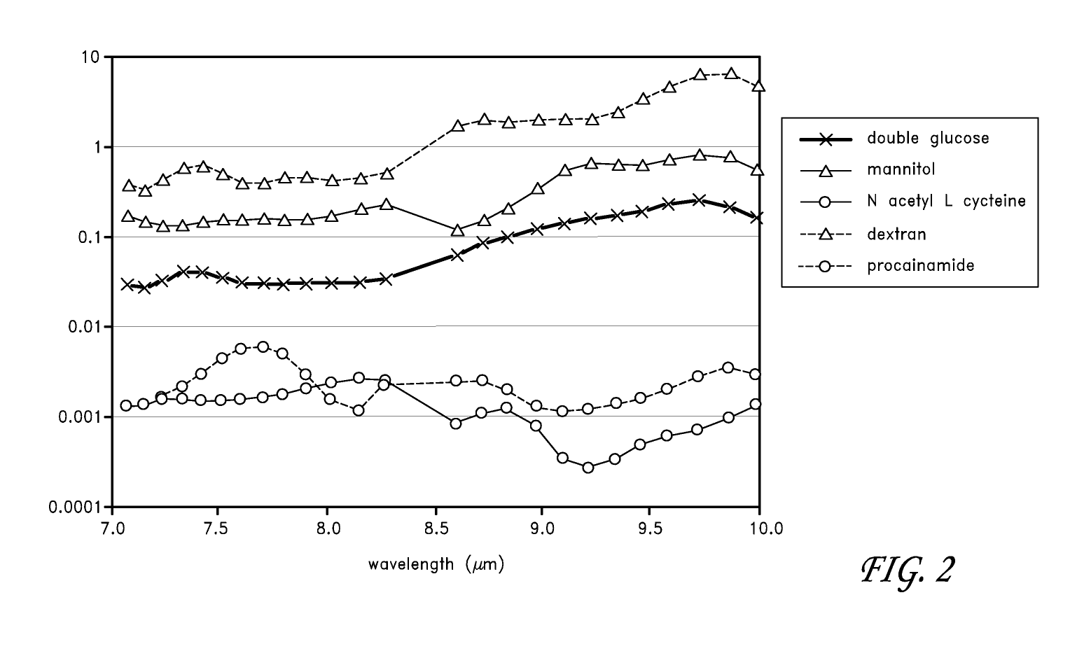

In addition to components naturally found in the blood, the ingestion or injection of some medicines or illicit drugs can result in very high and rapidly changing concentrations of exogenous interferents. This may result in difficulties in measuring analytes in blood of hospital or emergency room patients. An example of overlapping spectra of blood components and medicines is illustrated in FIG. 1 as the absorption coefficient at the same concentration and optical pathlength of pure glucose and three spectral interferents, specifically mannitol (chemical formula: hexane-1,2,3,4,5,6-hexaol), N acetyl L cysteine, dextran, and procainamide (chemical formula: 4-amino-N-(2-diethylaminoethyl)benzamid). FIG. 2 shows the logarithm of the change in absorption spectra from a Sample Population blood composition as a function of wavelength for blood containing additional likely concentrations of components, specifically, twice the glucose concentration of the Sample Population and various amounts of mannitol, N acetyl L cysteine, dextran, and procainamide. The presence of these components is seen to affect absorption over a wide range of wavelengths. It can be appreciated that the determination of the concentration of one species without a priori knowledge or independent measurement of the concentration of other species is problematic.

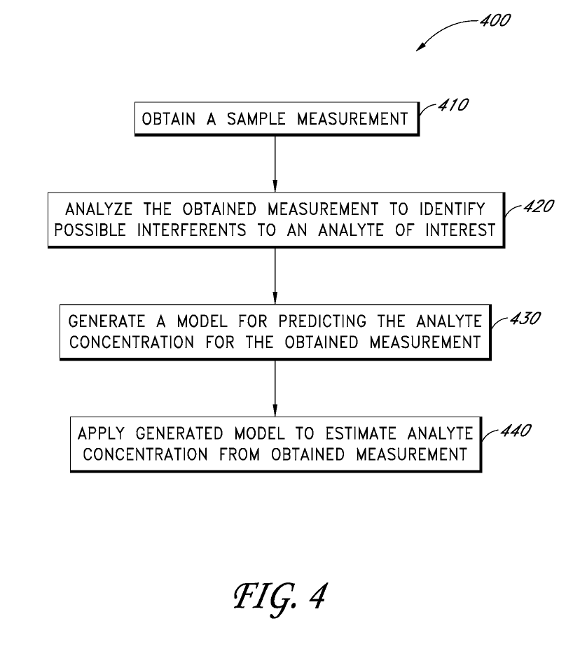

One method for estimating the concentration of an analyte in the presence of interferents is presented in flowchart 400 of FIG. 4 as Block 410 where a measurement of a sample is obtained, Block 420 where the obtained measurement data is analyzed to identify possible interferents to the analyte, Block 430 where a model is generated for predicting the analyte concentration in the presence of the identified possible interferents, and Block 440 where the model is used to estimate the analyte concentration in the sample from the measurement. In some embodiments, in Block 430 a model is generated where the error is reduced or minimized for the presence of the identified interferents that are not present in a general population of which the sample is a member.

An example embodiment of the method outlined in the flowchart 400 for the determination of an analyte from spectroscopic measurements will now be discussed. Further, this example embodiment is directed toward providing an estimate of the amount of glucose concentration in a blood sample S. It is to be recognized that this embodiment is illustrative and does not limit the scope of the inventions disclosed herein. In one embodiment, the measurement of Block 410 is an absorbance spectrum, Cₛ(λᵢ), of a measurement sample S that has, in general, one analyte of interest, glucose, and one or more interferents. In one embodiment, the methods include generating a calibration coefficient κ(λᵢ) that, when multiplied by the absorbance spectrum Cₛ(λᵢ), provides an estimate, gₑₛₜ=κ(λᵢ)Cₛ(λᵢ), of the glucose concentration gₛ.

As described below, in one embodiment of the method, Block 420 includes a statistical comparison of the absorbance spectrum of sample S with a spectrum of the Sample Population and combinations of individual Library Interferent spectra. After the analysis of Block 420, a list of Library Interferents that are possibly contained in sample S has been identified and includes, depending on the outcome of the analysis of Block 420, either no Library Interferents, or one or more Library Interferents. Block 430 then generates a large number of spectra using the large number of spectra of the Sample Population and their respective known analyte concentrations and known spectra of the identified Library Interferents. Block 430 then uses the generated spectra to generate a calibration coefficient matrix to convert a measured spectrum to an analyte concentration that is the least sensitive to the presence of the identified Library Interferents. Block 440 then applies the generated calibration coefficient to predict the glucose concentration in sample S.

As described above, in Block 410 the system obtains a measurement of a sample. For illustrative purposes, the measurement, Cₛ(λᵢ), is assumed to be a plurality of measurements at different wavelengths, or analyzed measurements, indicating the intensity of light that is absorbed by sample S. It is to be understood that spectroscopic measurements and computations may be performed in one or more domains including, but not limited to, the transmittance, absorbance and/or optical density domains. The measurement Cₛ(λᵢ) is an absorption, transmittance, optical density or other spectroscopic measurement of the sample at selected wavelength or wavelength bands. Such measurements may be obtained, for example, using analyte detection system 334. In general, sample S contains Type-A interferents, at concentrations preferably within the range of those found in the Sample Population.

In one embodiment, absorbance measurements are converted to pathlength normalized measurements. Thus, for example, the absorbance is converted to optical density by dividing the absorbance by the optical pathlength, L, of the measurement. In one embodiment, the pathlength L is measured from one or more absorption measurements on known compounds. Thus, in one embodiment, one or more measurements of the absorption through a sample S of water or saline solutions of known concentration are made, and the pathlength, L, is computed from the resulting absorption measurement(s). In another embodiment, absorption measurements are also obtained at portions of the spectrum that are not appreciably affected by the analytes and interferents, and the analyte measurement is supplemented with an absorption measurement at those wavelengths. For example, spectral measurements may be taken at an isosbestic point for an analyte and an interferent.

Embodiments of the method may determine which Library Interferents are present in the sample. For example, Block 420 indicates that the measurements are analyzed to identify possible interferents. In some systems using spectroscopic measurements, the determination of which Library Interferents are present is made by comparing, in the optical density domain, the obtained measurement to one or more interferent spectra. The comparison provides a list of interferents that may, or are likely to, be present in the sample. In one embodiment, several inputs are used to estimate a glucose concentration gₑₛₜ from a measured spectrum, Cₛ. The inputs include previously gathered spectrum measurement of samples that, like the measurement sample, include the analyte and combinations of possible interferents from the interferent library; and spectrum and concentration ranges for each possible interferent. More specifically, in certain embodiments, the inputs include:
Library of Interferent Data: Library of Interferent Data includes, for each of “M” interferents, the absorption spectrum of each interferent, IF={IF₁, IF₂, . . . , IF_{M}}, where m=1, 2, . . . , M; and a maximum concentration for each interferent, Tmax={Tmax₁, Tmax₂, . . . , Tmax_{M}}; and
Sample Population Data: Sample Population Data includes individual spectra of a statistically large population taken over the same wavelength range as the sample spectrum, Csᵢ, and an analyte concentration corresponding to each spectrum. As an example, if there are N Sample Population spectra, then the spectra can be represented as C={C₁, C₂, . . . , C_{N}}, where n=1, 2, . . . , N, and the analyte concentration corresponding to each spectrum can be represented as g={g₁, g₂, . . . , g_{N}}.

Advantageously, the Sample Population may be selected to not have any of the M interferents present in the Library of Interferents. The material sample may have interferents contained in the Sample Population and none, some, or all of the Library Interferents. Stated in terms of Type-A and Type-B interferents, the Sample Population has Type-A interferents and the material sample has Type-A and may have Type-B interferents. The Sample Population Data may be used to statistically quantify an expected range of spectra and analyte concentrations. Thus, for example, for a system 10 or 334 used to determine glucose in blood of a person having unknown spectral characteristics, the spectral measurements are preferably obtained from a statistical sample of the population.

### Interferent Determination

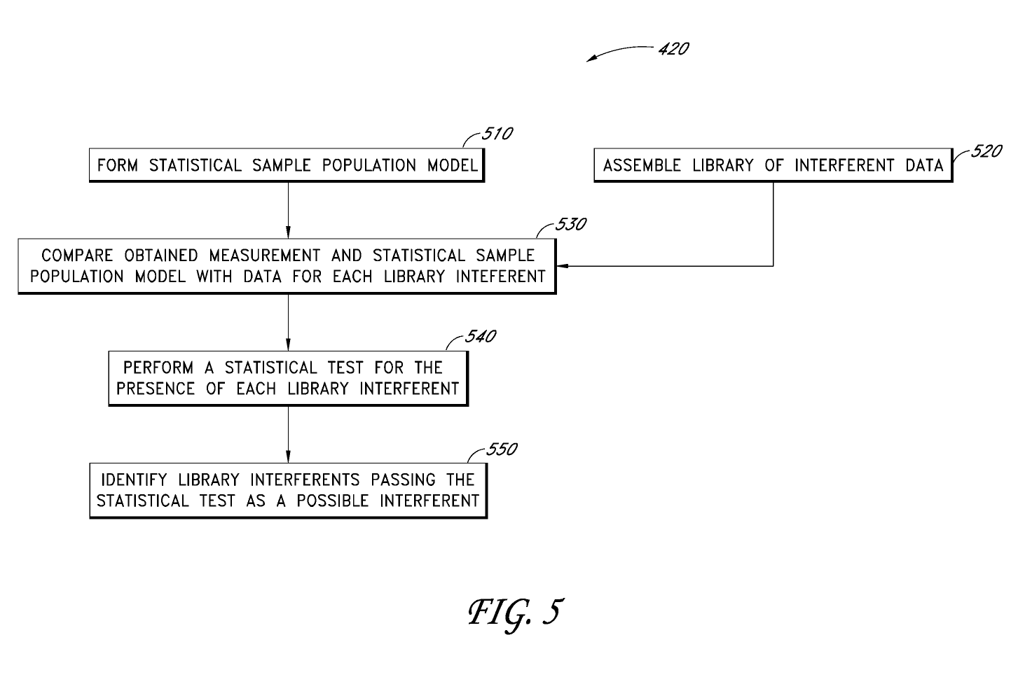

One embodiment of the method of Block 420 is shown in greater detail with reference to the flowchart of FIG. 5. The method includes forming a statistical Sample Population model (Block 510), assembling a library of interferent data (Block 520), comparing the obtained measurement and statistical Sample Population model with data for each interferent from an interferent library (Block 530), performing a statistical test for the presence of each interferent from the interferent library (Block 540), and identifying each interferent passing the statistical test as a possible Library Interferent (Block 550). The acts of Block 520 can be performed once or can be updated as necessary. The acts of Blocks 530, 540, and 550 can be performed sequentially for all interferents of the library, as shown in FIG. 5, or in other implementations, can be repeated sequentially for each interferent.

An embodiment of the methods of Blocks 510, 520, 530, 540, and 550 is now described for the example of identifying Library Interferents in a sample from a spectroscopic measurement using Sample Population Data and a Library of Interferent Data. Each Sample Population spectrum includes measurements (e.g., of optical density) taken on a sample in the absence of any Library Interferents and includes an associated known analyte concentration. A statistical Sample Population model is formed (Block 510) for the range of analyte concentrations by combining all Sample Population spectra to obtain a mean matrix and a covariance matrix for the Sample Population. Thus, for example, if each spectrum at N different wavelengths is represented by an N×1 matrix, C, then the mean spectrum, μ, is a N×1 matrix with the (e.g., optical density) value at each wavelength averaged over the range of spectra. The covariance matrix, V, is the expected value of the deviation between C and μ and can be written as V=E((C−μ)(C−μ)^{T}), where E(•) represents a statistical expectation operator and the superscript T represents transpose. The matrices μ and V are included in one model used to describe the statistical distribution of the Sample Population spectra. In other models, other statistical properties may be included additionally or alternatively. For example, some models include higher order matrices representing, e.g., skewness, kurtosis, etc. of the statistical distribution.

In Block 520, the system assembles Library Interferent information. A number M of possible interferents are selected, for example from possible medications or foods that might be ingested by the population of patients at issue. Spectra (e.g., in the absorbance, optical density, or transmission domains) are obtained. In addition, ranges of expected interferent concentrations in the blood, or other expected sample material, are estimated. For example, the concentration range for an interferent may be between 0 and a maximum concentration Tmax. The Library of Interferents may comprise, for each of M interferents, a spectrum IF and a maximum concentration Tmax. In some embodiments, the data in the Library is presented as a set of absorption spectrum for each interferent, IF={IF₁, IF₂, . . . , IF_{M}} and a set of maximum concentrations for each interferent, Tmax={Tmax₁, Tmax₂, . . . , Tmax_{M}). Advantageously, in some embodiments, the information in the Library is assembled once, stored, and accessed when needed.

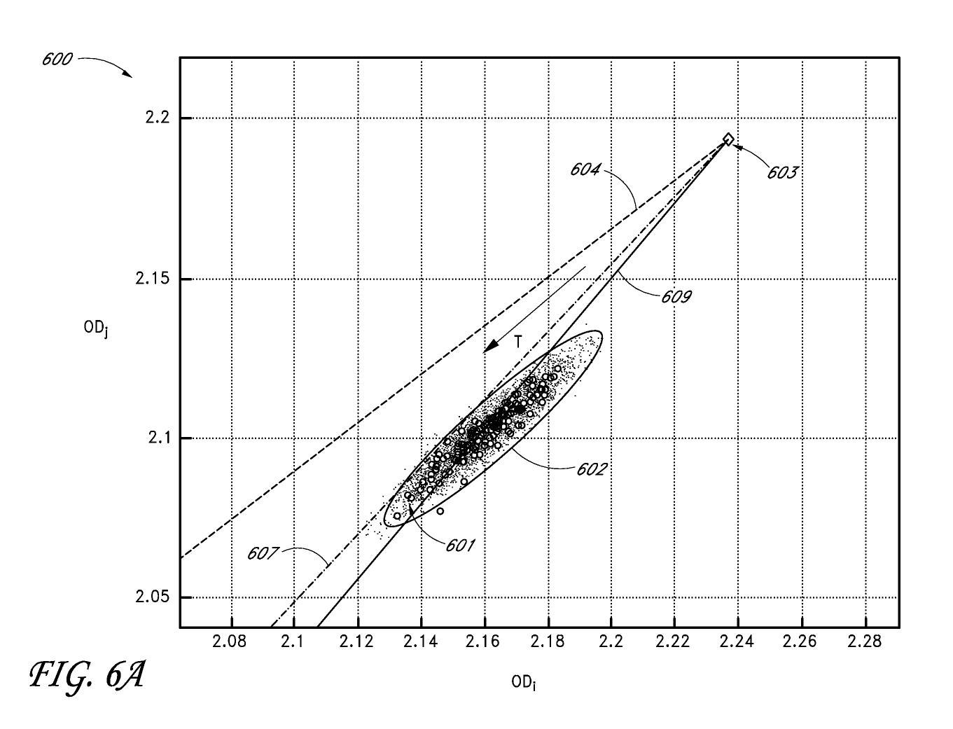

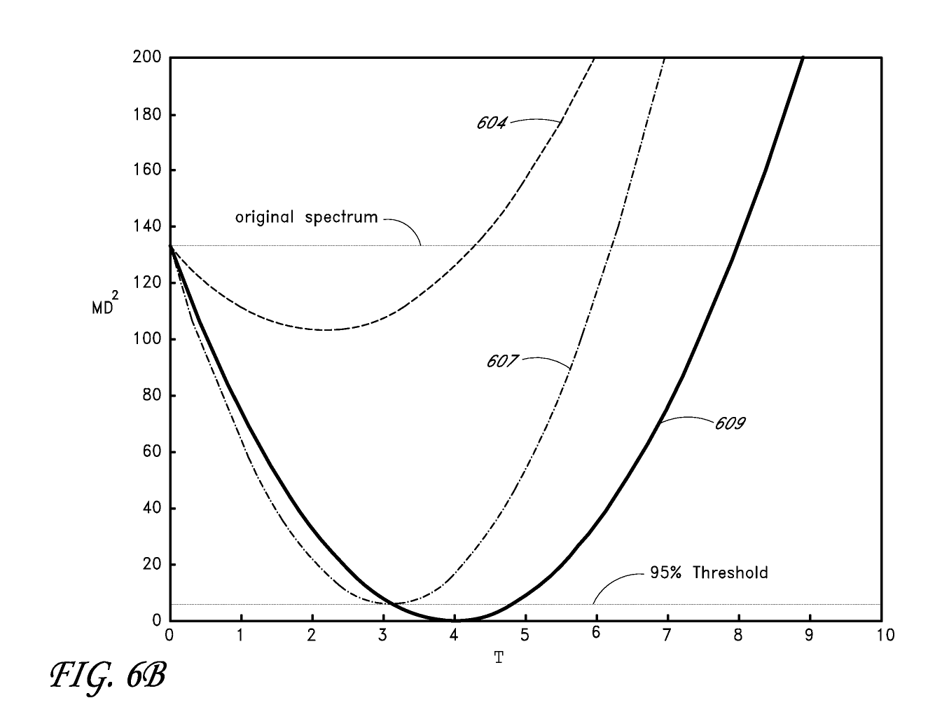

In Block 530, the obtained measurement data and the statistical Sample Population model are compared with data for each interferent from the Library of Interferents. In Block 540, the system performs a statistical test to determine the presence of each of the Library Interferents. In Block 550, the system identifies as a possible interferent to the analyte measurement any (or all) of the Library Interferents that pass the statistical test of Block 540. This interferent test will be described further below and with reference to FIGS. 6A and 6B.

One possible test for the presence of an interferent in a sample will now be described. The measured optical density spectrum, Cₛ, is modified for each Library Interferent by analytically subtracting the effect of the interferent, if present, on the measured spectrum. More specifically, the measured optical density spectrum, Cₛ, is modified, wavelength-by-wavelength, by subtracting an interferent optical density spectrum. For an interferent, M, having an absorption spectrum per unit of interferent concentration, IF_{M}, a modified spectrum, C′ₛ(T), is given by C′ₛ(T)=Cₛ−IF_{M} T, where T is the interferent concentration. The interferent concentration may be selected to be in a range from a minimum value, Tmin, to a maximum value, Tmax. The value of Tmin may be zero or, alternatively, be a value between zero and Tmax, such as some fraction of Tmax. In some embodiments, Tmin may be negative to reflect that the sample may include less of the interferent than is found in the Sample Population.

The Mahalanobis distance (MD) between the modified spectrum C′ₛ(T) and the statistical model (μ, V) of the Sample Population spectra is calculated from:

MD²(Cₛ−(T IF), μ; ρₛ)=(Cₛ−(T IFₘ)−μ)^{T}V⁻¹(Cₛ−(T IFₘ)−μ), Eq. (1)

where MD² (Cₛ−(T IF),μ; ρₛ) is also referred to herein as the “squared Mahalanobis distance,” or the “MD² score.” One possible test for the presence of interferent IF is to vary T from Tmin to Tmax (e.g., evaluate C′ₛ (T) over a range of values of T) and to determine whether the minimum MD² score in this range is in a particular interval and/or below a threshold. For example, the system may determine whether the minimum MD² score is sufficiently small relative to the quantiles of a χ² random variable with N degrees of freedom (N=number of wavelengths in the spectra). Although certain embodiments described herein use the MD² score, it is apparent that other embodiments may use the Mahalanobis distance MD (e.g., the square root of MD²) or any suitable function of the Mahalanobis distance. Also, other embodiments may utilize a different statistical measure of the difference between the spectra (or modified spectra) and the statistical model of the Sample Population Spectra such as, for example, Hotelling's T-square statistic, outlier analysis, regression techniques, and so forth.

FIG. 6A is a graph 600 illustrating an example of the acts of Blocks 530 and 540. The axes of the graph 600, ODᵢ and ODⱼ, are used to plot optical densities at two wavelengths (λᵢ, λⱼ) at which measurements are obtained. The points 601 are the measurements in the Sample Population distribution. The points 601 are clustered within an ellipse 602 that has been drawn to encircle the majority of points. The points 601 inside the ellipse 602 represent measurements in the absence of Library Interferents. In this example, point 603 is the sample measurement. Presumably, point 603 is outside of the spread of points 601 (indicated by the ellipse 602) due to the presence of one or more Library Interferents. Lines 604, 607, and 609 indicate the position of the sample point 603 in the graph, as the analyte concentration is adjusted for increasing concentrations, T, of three different Library Interferents, over the range from Tmin to Tmax. The three interferents of this example are referred to as interferent #1, interferent #2, and interferent #3. Specifically, lines 604, 607, and 609 are obtained by subtracting from the sample measurement an amount T of a Library Interferent (interferent #1, interferent #2, and interferent #3, respectively), and plotting the adjusted sample measurement, Cₛ′(T), for T in the range from Tmin to Tmax.

FIG. 6B is a graph illustrating the squared Mahalanobis distance, MD², plotted as a function of interferent concentration T for the lines 604, 607, and 609 in FIG. 6A. Referring to FIG. 6A, line 604 (in the direction indicated by an arrow referenced by T) reflects increasing concentrations of interferent #1 and only marginally approaches the points 601. FIG. 6B shows the value of MD² for line 604 decreases slightly and then increases with increasing interferent #1 concentration.

Referring back to FIG. 6A, the line 607 (in the direction of the arrow) reflects increasing concentrations of interferent #2 and approaches or passes through many of the points 601. FIG. 6B shows the value of MD² of the line 607 exhibits a large decrease at lower interferent #2 concentration and then increases. Referring back to FIG. 6A, the line 609 (in the direction of the arrow) has increasing concentrations of interferent #3 and approaches or passes through even more of the points 601 than the line 607. FIG. 6B shows the value of MD² of the line 609 exhibits a larger decrease than the line 607 at certain concentrations of the interferent #3.

In one embodiment, a threshold level of MD² is selected as an indication of the presence of a particular interferent. Thus, for example, FIG. 6B shows a line labeled “original spectrum” indicating the MD² score when no interferents are subtracted from the spectrum, and a line labeled “95% Threshold”, indicating the 95% quantile for a χ² distribution with N degrees of freedom (where N is the number of wavelengths represented in the spectra, in this case N=25). The 95% threshold represents the value that should exceed 95% of the values of the MD² score; in other words, MD² values below this threshold are relatively uncommon (e.g., occurring for only about 5% of the scores), and those far below the threshold should be quite rare. Of the three example interferents represented in FIGS. 6A and 6B, only interferent #3 has a value of MD² below the threshold. Thus, this example analysis of the sample indicates that interferent #3 is the most likely interferent present in the sample. Interferent #1 has its minimum MD² score significantly above the 95% threshold level and is therefore considered unlikely to be present. Interferent #2 just crosses below the 95% threshold, indicating that its presence is more likely than interferent #1, but less than interferent #3.

As described in more detail below, information related to the identified interferents may be used in generating a calibration coefficient that is relatively insensitive to a likely range of concentrations of the identified interferents. In addition to being used in certain embodiments of methods described below, the identification of the interferents (and their concentrations) in the sample may be of interest and may be provided in a manner that is useful to a medical practitioner. For example, in implementations of the system for a hospital based glucose monitor, the identified interferents may be reported on the display 141 and/or may be transmitted to a hospital computer via the communications link 216. The concentration of the identified interferents may be output on the display 141 or stored for later analysis. Any such information on the interferents may be stored by the system (e.g., in the memory 212 or any other suitable local and/or remote storage device) and may be tracked and reported (e.g., as a trend with time).

### Calculation of Calibration Coefficient

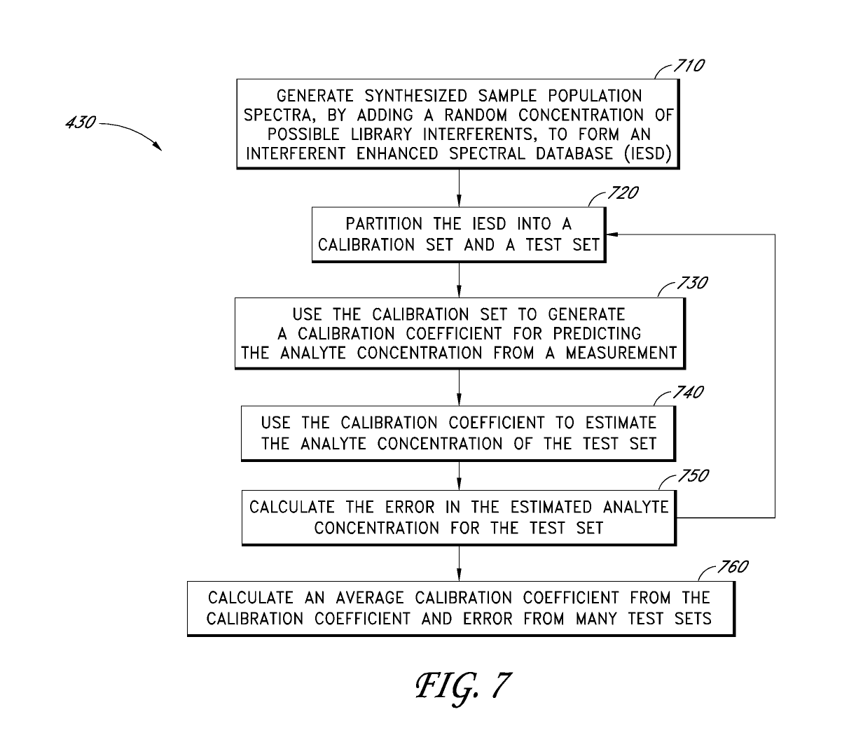

Once one or more Library Interferents are identified as being possibly present in the sample under analysis (Block 420), a calibration coefficient for estimating the concentration of analytes in the presence of the identified interferents is generated (Block 430). One embodiment of the acts of Block 430 is shown in the flowchart of FIG. 7. For example, in certain embodiments, in Block 710, the system generates synthesized Sample Population measurements; in Block 720, the synthesized Sample Population measurements are partitioned into a calibration set and a test set, in Block 730, the calibration set is used to generate a calibration coefficient, in Block 740, the calibration set is used to estimate the analyte concentration of the test set, in Block 750 errors in the estimated analyte concentration of the test set are calculated, and in Block 760 an average calibration coefficient is calculated.

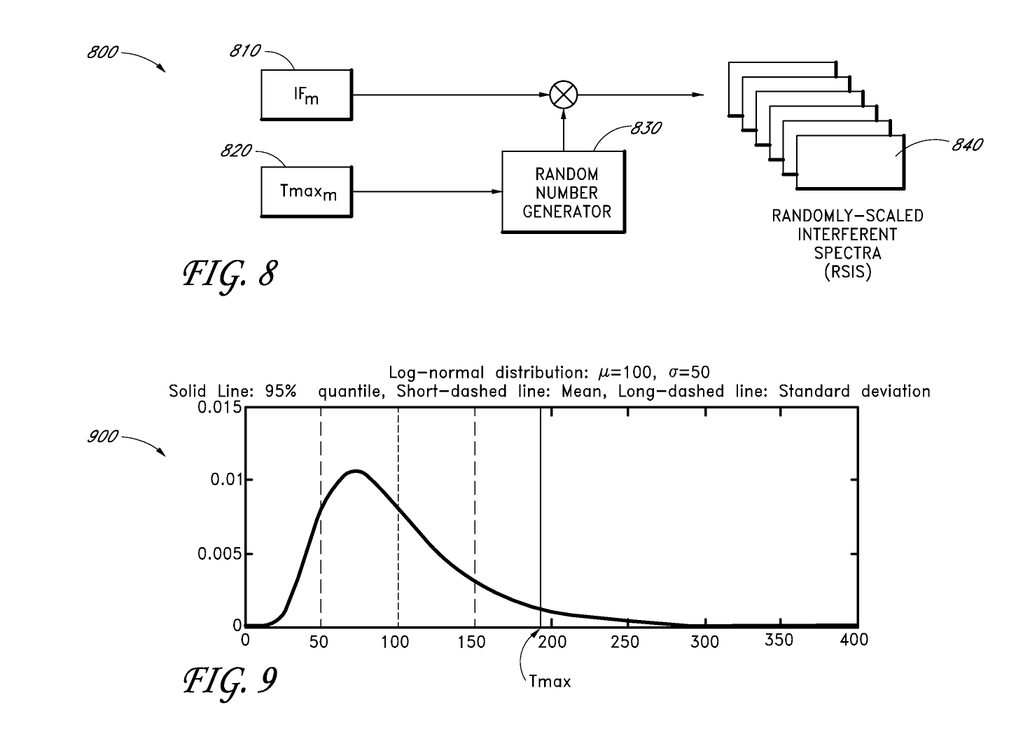

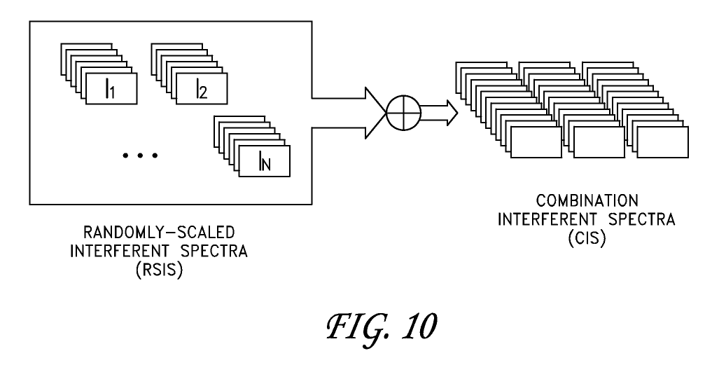

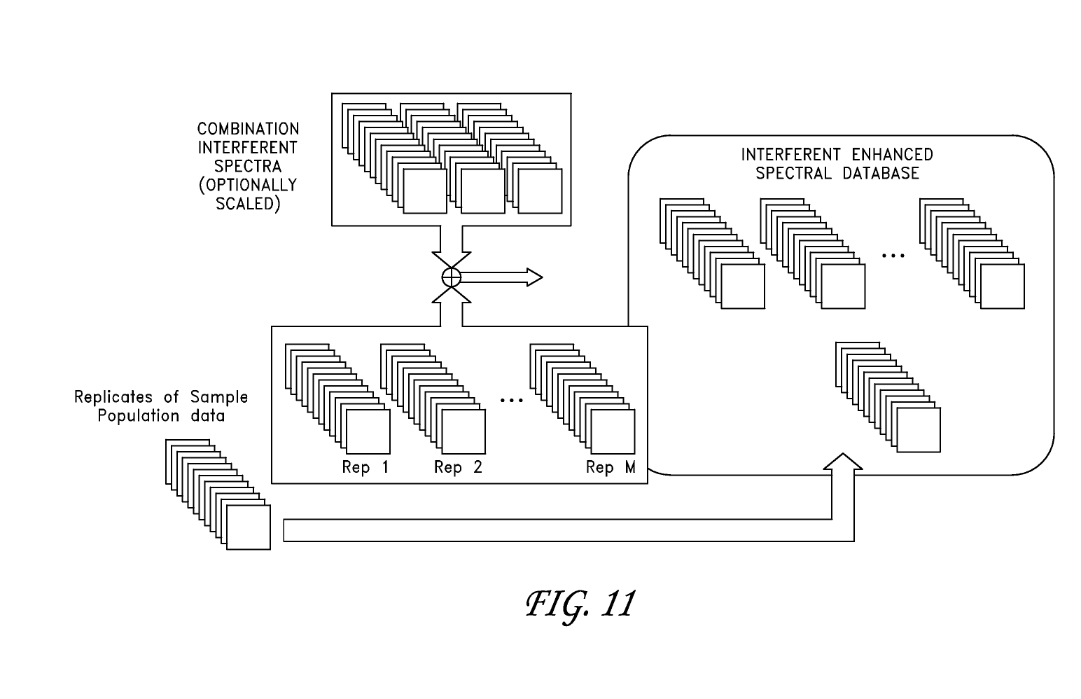

An example embodiment of the Blocks 710, 720, 730, 740, 750, and 760 will now be described. As shown in Block 710, the system generates synthesized Sample Population spectra by adding a random concentration of possible Library Interferents to each Sample Population spectrum. The spectra generated by the system in Block 710 are referred to herein as an Interferent-Enhanced Spectral Database, or IESD. As an example, the IESD can be formed according to the acts schematically illustrated in FIGS. 8-11. FIG. 8 is a schematic diagram 800 illustrating generation of Randomly-Scaled Single Interferent Spectra, or RSIS. FIG. 9 is a graph 900 of an example interferent concentration distribution function. FIG. 10 is a schematic diagram illustrating combination of the RSIS into Combination Interferent Spectra, or CIS. FIG. 11 is a schematic diagram illustrating combination of CIS and the Sample Population spectra into an IESD.

Examples of the acts that may be performed in Block 710 are further illustrated in FIGS. 8 and 9. As shown in schematic diagram 800 in FIG. 8, and in graph 900 in FIG. 9, a plurality of RSIS (Block 840) are formed by combinations of each previously identified Library Interferent having spectrum IFₘ (Block 810), multiplied by the maximum concentration Tmaxₘ (Block 820) that is scaled by a random number between zero and one (Block 830). An example probability distribution for the random numbers is shown in the graph 900 in FIG. 9. In this example, the probability distribution is a log-normal distribution with a mean of 100 and a standard deviation of 50. In FIG. 9, the location of the mean is indicated by a vertical short-dashed line, and the locations of the mean plus or minus one standard deviation are indicated by two vertical long-dashed lines. The 95% quantile of the distribution function is indicated by a vertical solid line. In one embodiment, the maximum concentration Tₘₐₓ is set to be at the 95% quantile of the random number distribution function. Although an example log-normal distribution is shown in FIG. 9, in other embodiments other random number distribution functions may be used such as, for example, a uniform distribution, a Gaussian distribution, a Poisson distribution, a chi-square distribution, etc.

In some embodiments, the RSIS are combined to produce a large population of interferent-only spectra, the Combination Interferent Spectra (CIS), for example as schematically illustrated in FIG. 10. In this example, the individual RSIS are combined independently and in random combinations to produce a large family of CIS, with each spectrum within the CIS including a random combination of RSIS, selected from the full set of identified Library Interferents. This embodiment of the method has been found to produce adequate variability with respect to each interferent, independently across separate interferents.

The Interferent Enhanced Spectral Database, IESD, may be formed by combining the CIS and replicates of the Sample Population spectra, as illustrated, for example, in the schematic diagram shown in FIG. 11. To account for the possibility that the Interferent Data and the Sample Population spectra may have been obtained at different pathlengths, the CIS can be scaled to the same pathlength. In some embodiments, the scaling of the CIS is performed by multiplying the CIS by a suitable scaling factor. As shown in the example in FIG. 11, the Sample Population database is replicated M times, where the choice of M may depend on the size of the database, the number of interferents to be analyzed, etc. The IESD includes M copies of each of the Sample Population spectra, where one copy is the original Sample Population Data, and the remaining M−1 copies each have a random CIS spectra included. Each of the IESD spectra has an associated known analyte concentration from the Sample Population spectra used to form the particular IESD spectrum.

In one embodiment, a 10-fold replication of the Sample Population database is used for 130 Sample Population spectra obtained from 58 different individuals and 18 Library Interferents. If there is greater spectral variety among the Library Interferent spectra, the formation of the IESD may utilize a smaller replication factor. If there is a greater number of Library Interferents, the formation of the IESD may utilize a larger replication factor.

As shown in FIG. 7, the Blocks 720, 730, 740, and 750 may be executed to repeatedly combine different ones of the spectra of the IESD to statistically average out the effect of the identified Library Interferents. As shown in Block 720, the IESD may be partitioned into two subsets: a calibration set and a test set. As described below, the repeated partitioning of the IESD into different calibration sets and test sets may improve the statistical significance of the calibration coefficient determined in Block 760. In one embodiment, the calibration set includes a random selection of some of the IESD spectra, and the test set includes the remaining unselected IESD spectra. In a preferred embodiment, the calibration set includes approximately two-thirds of the IESD spectra.

In an alternative embodiment, Blocks 720, 730, 740, and 750 are combined and a single calculation of an average calibration coefficient is performed using all available data.

Continuing in Block 730, the calibration set is used to generate a calibration coefficient for predicting the analyte concentration from a sample measurement. For the case of glucose concentration determined from spectroscopic absorption measurements, a glucose absorption spectrum is denoted as α_{G}. In an embodiment, the system may determine the calibration coefficient as follows. Using the calibration set having calibration spectra C={C₁, C₂, . . . , Cn} and corresponding known glucose concentration values G={g₁, g₂, . . . , gₙ} glucose-free spectra C′={C′₁, C′₂, . . . , C′ₙ} can be calculated as C=C′−αG, e.g., C′ⱼ=Cⱼ−αG gⱼ. The calibration coefficient, κ, may be calculated in certain embodiments from C′ and α_{G}, as follows:
1) C′ is decomposed into C′=A_{C′}Δ_{C′}B_{C′}, using, e.g., a singular value decomposition algorithm, where A_{C′} provides an orthonormal basis of column space, or span, of C′;
2) A_{C′} is truncated to avoid overfitting to a particular column rank r, based on the sizes of the diagonal entries of Δ (e.g., the singular values of C′). The selection of the rank r may involve a trade-off between precision and stability of the calibration, with a larger value of r resulting in a more precise but less stable solution. In one embodiment, each spectrum C includes 25 wavelengths, and the rank r ranges from 15 to 19;
3) The first r columns of A_{C′} are taken as an orthonormal basis of span(C′);
4) The projection from the background is determined as the product P_{C′}=A_{C′}A_{C′}^{T}, which is the orthogonal projection onto the span of C′. The complementary, or nulling, projection P_{C′}^{⊥}=1−P_{C′}, which forms the projection onto the complementary subspace C′^{⊥}, is calculated; and
5) The calibration coefficient vector κ is calculated by applying the nulling projection to the absorption spectrum of the analyte of interest: κ_{RAW}=P_{c′}^{⊥}α_{G} and normalizing: κ=κ_{RAW}/<κ_{RAW}, α_{G}>, where the angle brackets <,> denote the standard inner (or dot) product of vectors. The normalized calibration coefficient produces a unit response for a unit α_{G} spectral input for one particular calibration set.

In certain embodiments, the calibration coefficient is used to estimate the analyte concentration in the test set (Block 740). For example, each spectrum of the test set has an associated known glucose concentration based on the Sample Population spectra used to generate the test set. Each spectrum of the test set is multiplied by the calibration vector K (determined in Block 730) to calculate an estimated glucose concentration. The error between the calculated and known glucose concentration is then determined by the system in Block 750. In some embodiments, the measure of the error can include a weighted value averaged over the entire test set according to, for example, weighting functions that are inversely proportional to the root-mean-square (rms) error (e.g., 1/rms²).

Blocks 720, 730, 740, and 750 may be repeated for many different random combinations of calibration sets. For example, Blocks 720, 730, 740, and 750 can be repeated hundreds to thousands of times. In Block 760, an average calibration coefficient is calculated from the calibration and error from the many calibration and test sets. In some embodiments, the average calibration is computed as weighted average calibration vector. For example, in one embodiment, the weighting is in proportion to a normalized rms, and the average calibration coefficient is determined as κₐᵥₑ=κ*rms²/Σ(rms²), where the sum is over all tests.

Returning to the flowchart 400 shown in FIG. 4, in Block 440 the system applies the average calibration coefficient κₐᵥₑ to the sample spectrum obtained in Block 410 to estimate the analyte concentration. In certain embodiments, the estimated analyte concentration is calculated from the average calibration coefficient and the spectrum Cₛ obtained from the sample according to: gₑₛₜ=κₐᵥₑCₛ.

Accordingly, one possible embodiment of a method of computing a calibration coefficient based on identified interferents comprises the following:
1. Generate synthesized Sample Population spectra by adding the RSIS to raw (e.g., interferent-free) Sample Population spectra, thereby forming an Interferent Enhanced Spectral Database (IESD). Each spectrum of the IESD is synthesized from one spectrum of the Sample Population, and thus each spectrum of the IESD has at least one associated known analyte concentration
2. Separate the spectra of the IESD into a calibration set of spectra and a test set of spectra
3. Generate a calibration coefficient for the calibration set based on the calibration set spectra and their associated known correct analyte concentrations (e.g., using the matrix manipulation described above)
4. Use the calibration coefficient generated in step 3 to calculate the error in the corresponding test set as follows (repeat for each spectrum in the test set):
   a. Multiply (the selected test set spectrum)×(average calibration coefficient generated in step 3) to generate an estimated analyte (e.g., glucose) concentration
   b. Evaluate the difference between this estimated analyte concentration and the known analyte concentration associated with the selected test spectrum to generate an error associated with the selected test spectrum
5. Average the errors calculated in step 4 to arrive at a weighted or average error for the current calibration set-test set pair
6. Repeat steps 2 through 5 n times, resulting in n calibration coefficients and n average errors
7. Compute a “grand average” error from the n average errors and an average calibration coefficient from the n calibration coefficients (preferably using weighted averages wherein the largest average errors and calibration coefficients are discounted), to arrive at a calibration coefficient which is minimally sensitive to the effect of the identified interferents

### Example 1

One example of certain methods disclosed herein is illustrated with reference to the detection of glucose in blood using mid-infrared absorption spectroscopy. Table 1 lists 10 Library Interferents (each having absorption features that overlap with glucose) and the corresponding maximum concentration of each Library Interferent. Table 1 also lists a Glucose Sensitivity to Interferent without and with training. The Glucose Sensitivity to Interferent is the calculated change in estimated glucose concentration for a unit change in interferent concentration. For a highly glucose selective analyte detection technique, the Glucose Sensitivity to Interferent value is zero. The Glucose Sensitivity to Interferent without training is the Glucose Sensitivity to Interferent where the calibration has been determined using the methods above without any identified interferents. The Glucose Sensitivity to Interferent with training is the Glucose Sensitivity to Interferent where the calibration has been determined using the methods above with the appropriately identified interferents. In this case, the least improvement (in terms of reduction in sensitivity to an interferent) occurs for urea, with a factor of 6.4 lower sensitivity. Three other interferents show a factor of about 60 to 80 in improvement. The remaining six interferents all have seen sensitivity factors reduced by over 100 and in one case there is a sensitivity reduction by over 1600. The decreased Glucose Sensitivity to Interferent with training indicates that the disclosed methods are effective at producing a calibration coefficient that is selective to glucose in the presence of interferents.

TABLE 1
Rejection of 10 interfering substances

| | | Glucose | Glucose |
|---|---|---|---|
| | Maximum | Sensitivity to | Sensitivity to |
| Library | Concentration | Interferent | Interferent |
| Interferent | (mg/dL) | w/o training | w/training |
| Sodium Bicarbonate | 103 | 0.330 | 0.0002 |
| Urea | 100 | −0.132 | 0.0206 |
| Magnesium Sulfate | 0.7 | 1.056 | −0.0016 |
| Naproxen | 10 | 0.600 | −0.0091 |
| Uric Acid | 12 | −0.557 | 0.0108 |
| Salicylate | 10 | 0.411 | −0.0050 |
| Glutathione | 100 | 0.041 | 0.0003 |
| Niacin | 1.8 | 1.594 | −0.0086 |
| Nicotinamide | 12.2 | 0.452 | −0.0026 |
| Chlorpropamide | 18.3 | 0.334 | 0.0012 |

### Example 2

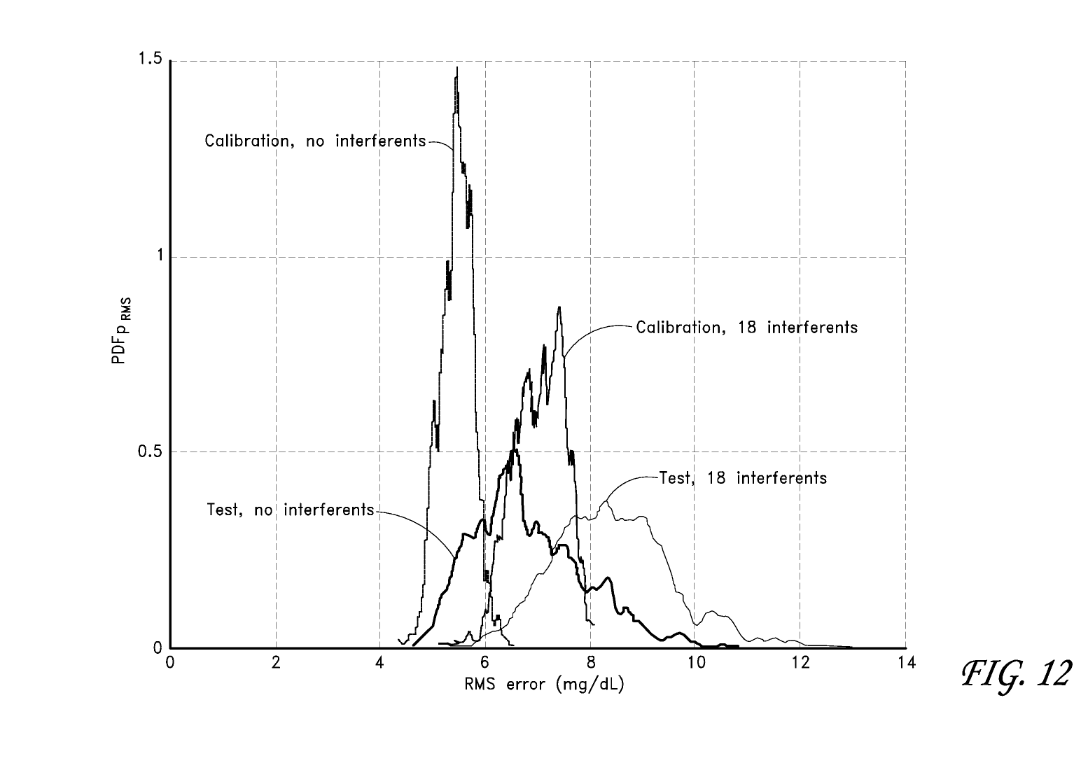

Another example illustrates the effect of the methods for 18 interferents. Table 2 lists of 18 interferents and maximum concentrations that were modeled for this example, and the glucose sensitivity to the interferent without and with training. The table summarizes the results of a series of 1000 calibration and test simulations that were performed both in the absence of the interferents, and with all interferents present. FIG. 12 shows the distribution of the root-mean-square (rms) error in the glucose concentration estimation for 1000 trials. While a number of substances show significantly less sensitivity (sodium bicarbonate, magnesium sulfate, tolbutamide), others show increased sensitivity (ethanol, acetoacetate), as listed in Table 2. The curves in FIG. 12 are for calibration set and the test set both without any interferents and with all 18 interferents. The interferent produces a degradation of performance, as can be seen by comparing the calibration and test curves of FIG. 12. Thus, for example, the peaks in the depicted distributions appear to be shifted by about 2 mg/dL, and the width of the distributions is increased slightly. The reduction in height of the peaks is due to the spreading of the distributions, resulting in a modest degradation in performance.

TABLE 2
List of 18 Library Interferents with maximum concentrations
and Glucose Sensitivity with respect to interferents,
with and without training

| | | | Glucose | Glucose |
|---|---|---|---|---|
| | | | Sensitivity to | Sensitivity to |
| | Library | Conc. | Interferent | Interferent |
| | Interferent | (mg/dL) | w/o training | w/training |
| 1 | Urea | 300 | −0.167 | −0.100 |
| 2 | Ethanol | 400.15 | −0.007 | −0.044 |
| 3 | Sodium Bicarbonate | 489 | 0.157 | −0.093 |
| 4 | Acetoacetate Li | 96 | 0.387 | 0.601 |
| 5 | Hydroxybutyric Acid | 465 | −0.252 | −0.101 |
| 6 | Magnesium Sulfate | 29.1 | 2.479 | 0.023 |
| 7 | Naproxen | 49.91 | 0.442 | 0.564 |
| 8 | Salicylate | 59.94 | 0.252 | 0.283 |
| 9 | Ticarcillin Disodium | 102 | −0.038 | −0.086 |
| 10 | Cefazolin | 119.99 | −0.087 | −0.006 |
| 11 | Chlorpropamide | 27.7 | 0.387 | 0.231 |
| 12 | Nicotinamide | 36.6 | 0.265 | 0.366 |
| 13 | Uric Acid | 36 | −0.641 | −0.712 |
| 14 | Ibuprofen | 49.96 | −0.172 | −0.125 |
| 15 | Tolbutamide | 63.99 | 0.132 | 0.004 |
| 16 | Tolazamide | 9.9 | 0.196 | 0.091 |
| 17 | Bilirubin | 3 | −0.391 | −0.266 |
| 18 | Acetaminophen | 25.07 | 0.169 | 0.126 |

### Example 3

In a third example, certain methods disclosed herein were tested for measuring glucose in blood using mid-infrared absorption spectroscopy in the presence of four interferents not normally found in blood (Type-B interferents) and that may be common for patients in hospital intensive care units (ICUs). The four Type-B interferents are mannitol, dextran, n-acetyl L cysteine, and procainamide.

Of the four Type-B interferents, mannitol and dextran have the potential to interfere substantially with the estimation of glucose: both are spectrally similar to glucose (see FIG. 1), and the dosages employed in ICUs are very large in comparison to typical glucose levels. Mannitol, for example, may be present in the blood at concentrations of 2500 mg/dL, and dextran may be present at concentrations in excess of 5000 mg/dL. For comparison, typical plasma glucose levels are on the order of 100-200 mg/dL. The other Type-B interferents, n-acetyl L cysteine and procainamide, have spectra that are quite unlike the glucose spectrum.

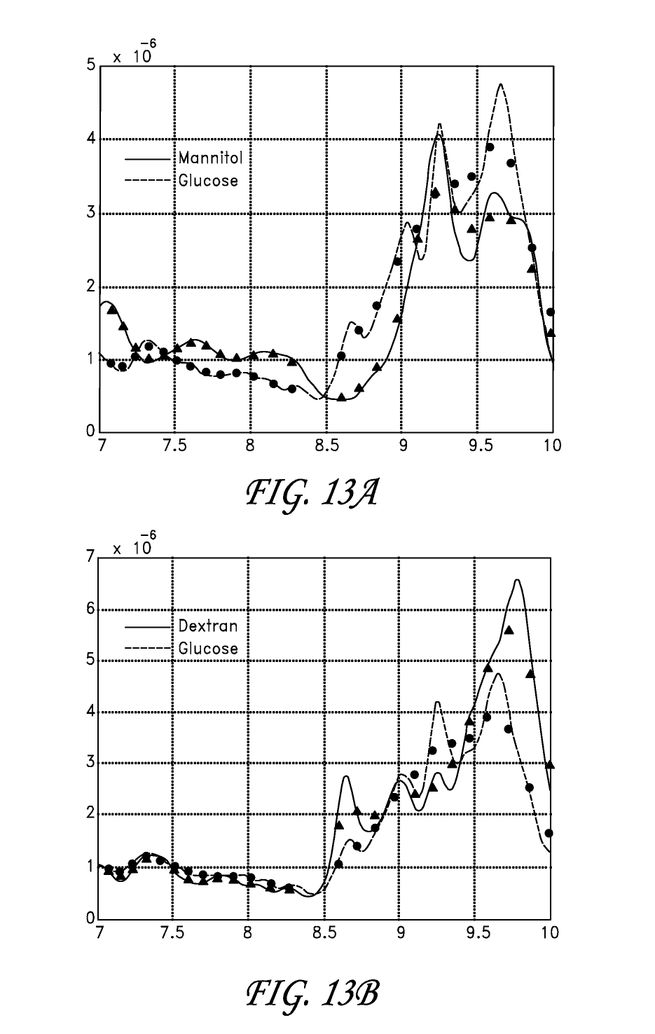

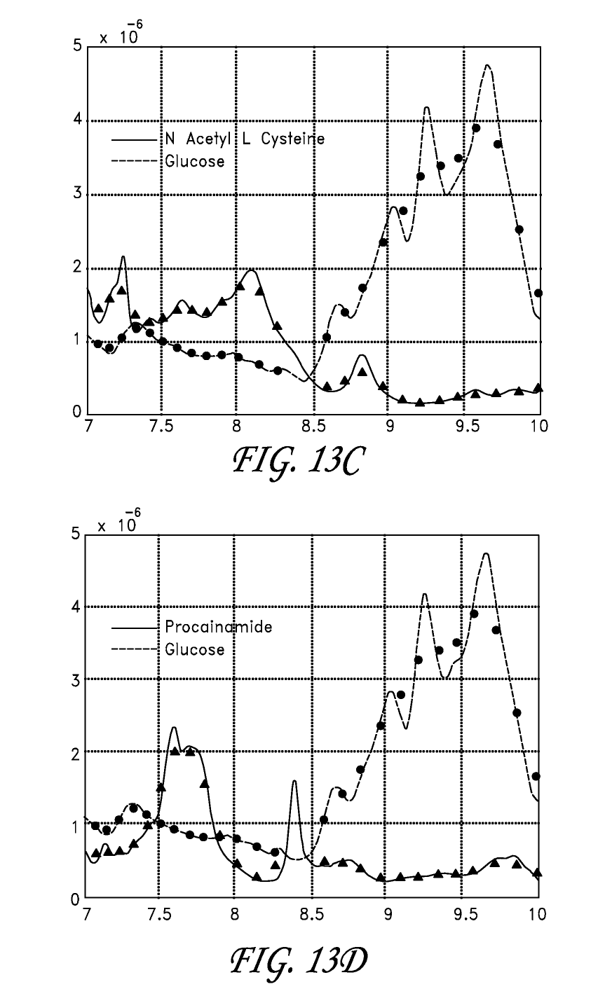

FIGS. 13A, 13B, 13C, and 13D each have a graph showing a comparison of the absorption spectrum of glucose with different interferents. The absorption spectra were taken using two different techniques: a Fourier Transform Infrared (FTIR) spectrometer having an interpolated resolution of 1 cm⁻¹ (solid lines with triangles) and using 25 finite-bandwidth IR filters having a Gaussian profile and full-width half-maximum (FWHM) bandwidth of 28 cm⁻¹ corresponding to a bandwidth that varies from 140 nm at 7.08 μm, up to 279 nm at 10 μm (dashed lines with circles). Specifically, the figures show a comparison of glucose with mannitol (FIG. 13A), with dextran (FIG. 13B), with n-acetyl L cysteine (FIG. 13C), and with procainamide (FIG. 13D), at a concentration level of 1 mg/dL and path length of 1 μm. The horizontal axes in FIGS. 13A-13D have units of wavelength in microns (μm), ranging from 7 μm to 10 μm, and the vertical axes have arbitrary units.

The central wavelength of the data obtained using filter is indicated in FIGS. 13A, 13B, 13C, and 13D by the circles along each dashed curve, and corresponds to the following wavelengths, in microns: 7.082, 7.158, 7.241, 7.331, 7.424, 7.513, 7.605, 7.704, 7.800, 7.905, 8.019, 8.150, 8.271, 8.598, 8.718, 8.834, 8.969, 9.099, 9.217, 9.346, 9.461, 9.579, 9.718, 9.862, and 9.990. The effect of the bandwidth of the filters on the spectral features can be seen in FIGS. 13A-13D as the decrease in the sharpness of spectral features on the solid curves and the relative absence of sharp features on the dashed curves.

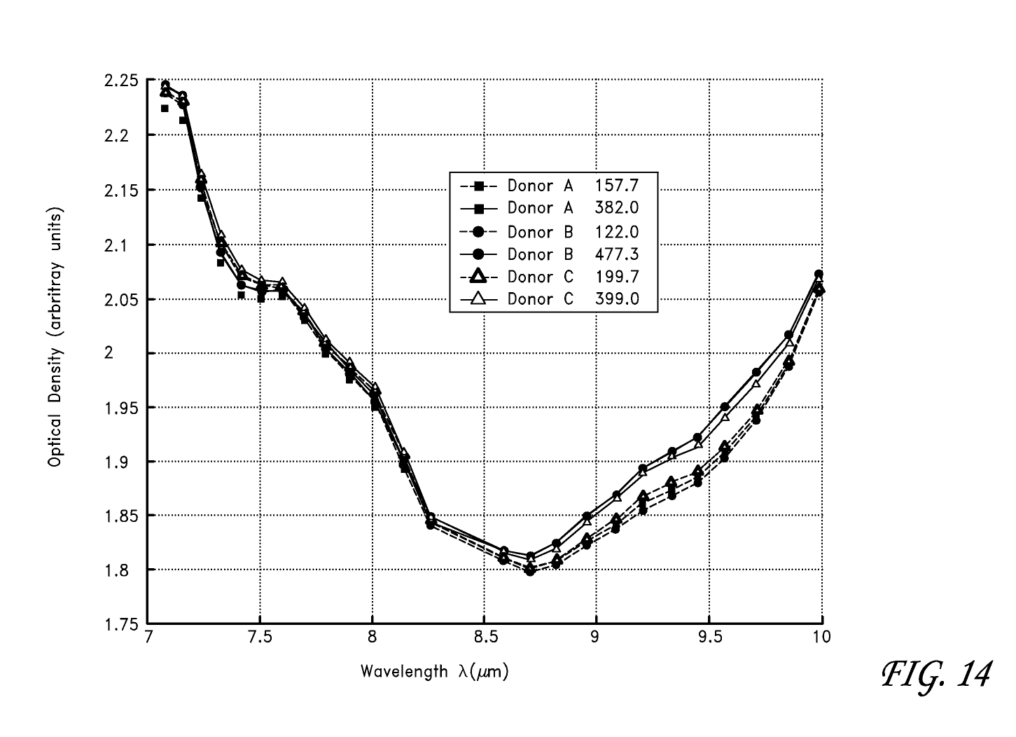

FIG. 14 shows a graph of the blood plasma spectra for 6 blood samples taken from three donors in arbitrary units for a wavelength range from 7 μm to 10 μm, where the symbols on the curves indicate the central wavelengths of the 25 filters. The 6 blood samples do not contain any mannitol, dextran, n-acetyl L cysteine, and procainamide—the Type-B interferents of this Example, and are thus a Sample Population. Three donors (indicated as donors A, B, and C) provided blood at different times, resulting in different blood glucose levels, shown in the graph legend in mg/dL as measured using a YSI Biochemistry Analyzer (YSI Incorporated, Yellow Springs, Ohio). The path length of these samples, estimated at 36.3 μm by analysis of the spectrum of a reference scan of saline in the same cell immediately prior to measurement of each sample spectrum, was used to normalize these measurements. The pathlength was taken into account in the computation of the calibration coefficient vectors, and the application of the computed calibration vectors to spectra obtained from other equipment advantageously may use a similar pathlength normalization process to obtain results having reliability.

Random amounts of each Type-B interferent of this Example were added to the spectra to produce mixtures that, for example, could make up an Interferent Enhanced Spectral Database (IESD). Each of the Sample Population spectra was combined with a random amount of a single interferent, as indicated in Table 3. Table 3 lists an index number N, the Donor, the glucose concentration (GLU), interferent concentration (conc(IF)), and the interferent for each of 54 spectra. The parameters shown in Table 3 were used to form combined spectra that include each of the 6 plasma spectra combined with 2 levels of each of the 4 interferents.

TABLE 3
Interferent Enhanced Spectral Database (IESD) for Example 3

| N | Donor | GLU | conc(IF) | IF |
|---|---|---|---|---|
| 1 | A | 157.7 | | N/A |
| 2 | A | 382 | | N/A |
| 3 | B | 122 | | N/A |
| 4 | B | 477.3 | | N/A |
| 5 | C | 199.7 | | N/A |
| 6 | C | 399 | | N/A |
| 7 | A | 157.7 | 1001.2 | Mannitol |
| 8 | A | 382 | 2716.5 | Mannitol |
| 9 | A | 157.7 | 1107.7 | Mannitol |
| 10 | A | 382 | 1394.2 | Mannitol |
| 11 | B | 122 | 2280.6 | Mannitol |
| 12 | B | 477.3 | 1669.3 | Mannitol |
| 13 | B | 122 | 1710.2 | Mannitol |
| 14 | B | 477.3 | 1113.0 | Mannitol |
| 15 | C | 199.7 | 1316.4 | Mannitol |
| 16 | C | 399 | 399.1 | Mannitol |
| 17 | C | 199.7 | 969.8 | Mannitol |
| 18 | C | 399 | 2607.7 | Mannitol |
| 19 | A | 157.7 | 8.8 | N Acetyl L Cysteine |
| 20 | A | 382 | 2.3 | N Acetyl L Cysteine |
| 21 | A | 157.7 | 3.7 | N Acetyl L Cysteine |
| 22 | A | 382 | 8.0 | N Acetyl L Cysteine |
| 23 | B | 122 | 3.0 | N Acetyl L Cysteine |
| 24 | B | 477.3 | 4.3 | N Acetyl L Cysteine |
| 25 | B | 122 | 8.4 | N Acetyl L Cysteine |
| 26 | B | 477.3 | 5.8 | N Acetyl L Cysteine |
| 27 | C | 199.7 | 7.1 | N Acetyl L Cysteine |
| 28 | C | 399 | 8.5 | N Acetyl L Cysteine |
| 29 | C | 199.7 | 4.4 | N Acetyl L Cysteine |
| 30 | C | 399 | 4.3 | N Acetyl L Cysteine |
| 31 | A | 157.7 | 4089.2 | Dextran |
| 32 | A | 382 | 1023.7 | Dextran |
| 33 | A | 157.7 | 1171.8 | Dextran |
| 34 | A | 382 | 4436.9 | Dextran |
| 35 | B | 122 | 2050.6 | Dextran |
| 36 | B | 477.3 | 2093.3 | Dextran |
| 37 | B | 122 | 2183.3 | Dextran |
| 38 | B | 477.3 | 3750.4 | Dextran |
| 39 | C | 199.7 | 2598.1 | Dextran |
| 40 | C | 399 | 2226.3 | Dextran |
| 41 | C | 199.7 | 2793.0 | Dextran |
| 42 | C | 399 | 2941.8 | Dextran |
| 43 | A | 157.7 | 22.5 | Procainamide |
| 44 | A | 382 | 35.3 | Procainamide |
| 45 | A | 157.7 | 5.5 | Procainamide |
| 46 | A | 382 | 7.7 | Procainamide |
| 47 | B | 122 | 18.5 | Procainamide |
| 48 | B | 477.3 | 5.6 | Procainamide |
| 49 | B | 122 | 31.8 | Procainamide |
| 50 | B | 477.3 | 8.2 | Procainamide |
| 51 | C | 199.7 | 22.0 | Procainamide |
| 52 | C | 399 | 9.3 | Procainamide |
| 53 | C | 199.7 | 19.7 | Procainamide |
| 54 | C | 399 | 12.5 | Procainamide |

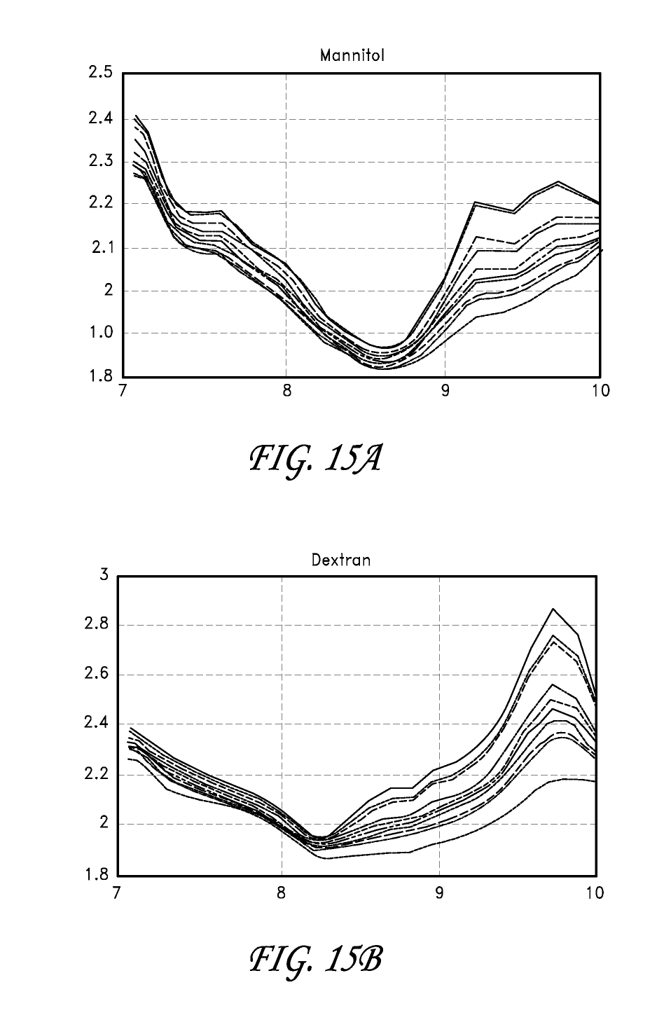

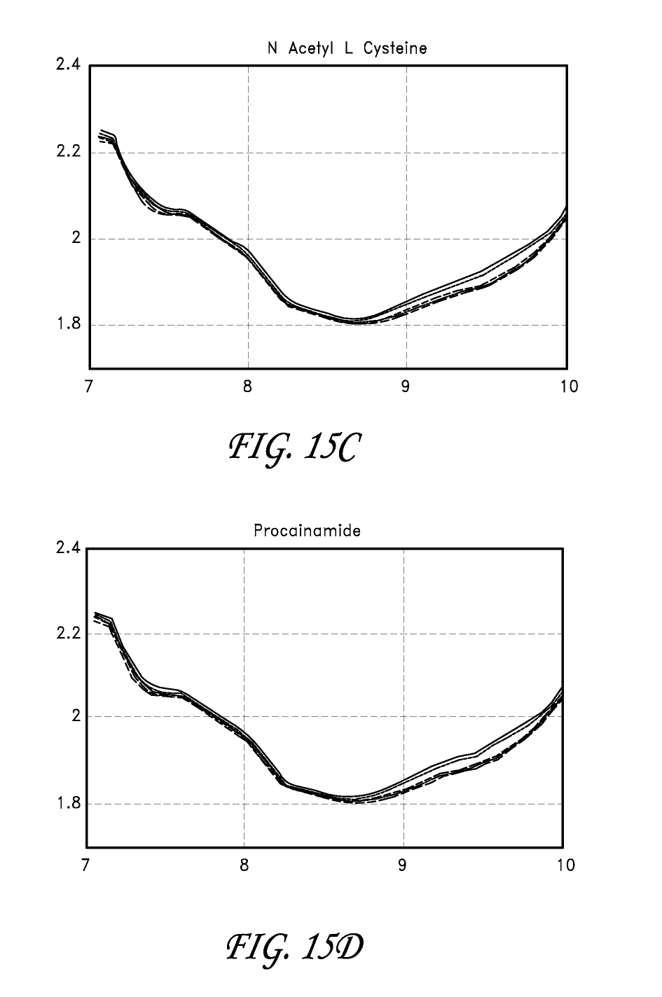

FIGS. 15A-15D show spectra from the IESD having random amounts of mannitol (FIG. 15A), dextran (FIG. 15B), n-acetyl L cysteine (FIG. 15C), and procainamide (FIG. 15D), normalized to concentration levels of 1 mg/dL and path lengths of 1 μm.

Calibration coefficient vectors were generated using the spectra of FIGS. 15A-15D, according to the methods described with reference to Block 420. As discussed above, many of the methods disclosed herein enable the estimation of an analyte concentration in the presence of interferents that are present in both the Sample Population and the measurement sample (Type-A interferents). Accordingly, in certain embodiments, the processor does not correct the spectra for interferents present in the Sample Population and the measurement sample before calculating the calibration coefficient.

In some embodiments, the spectra can be adjusted to remove the effects of one or more Type-A interferents (e.g., water) on the spectra. In Example 3, water-free spectra were generated by spectral subtraction of the water that was present in the sample. Adjusting spectra to remove the effects of one or more Type-A interferent is optional and, in some cases, advantageously may increase the accuracy of the method.

As described above, the system may use the calibration vector to compute an analyte concentration by evaluating a dot-product of the calibration vector with a vector representing spectral absorption values for the filters used in processing the reference spectra. Optionally, the spectral absorption values may be pathlength normalized.

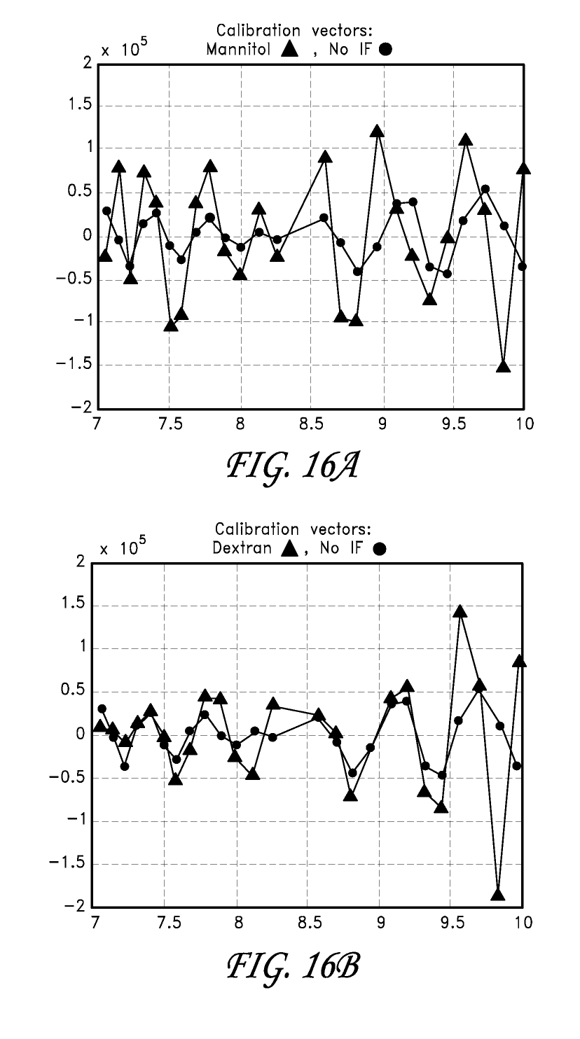

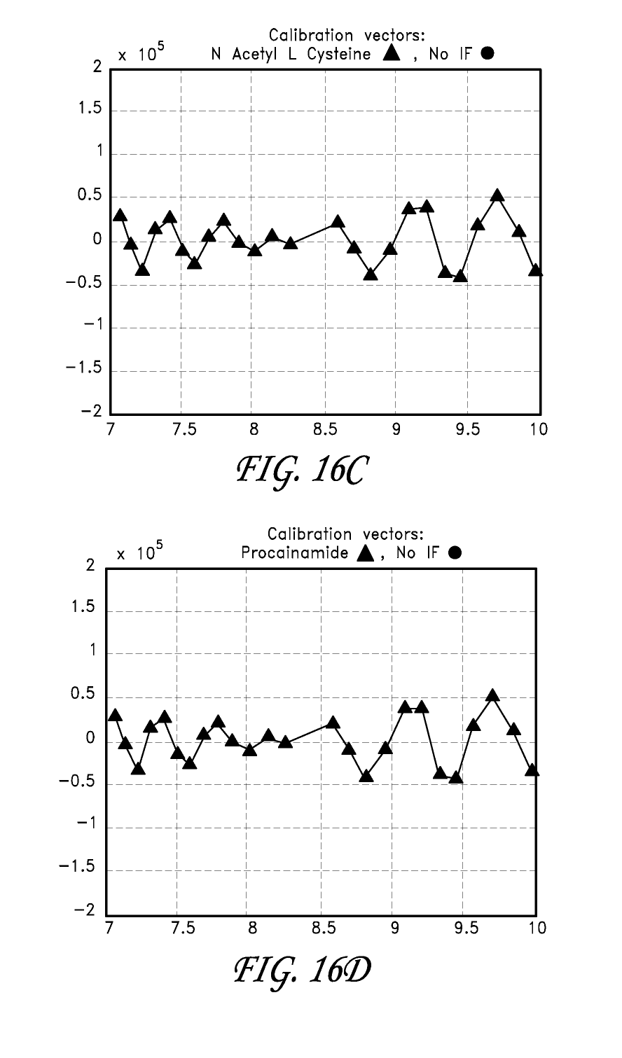

Graphs of the computed calibration coefficient vectors are shown in FIGS. 16A-16D for mannitol (FIG. 16A), dextran (FIG. 16B), n-acetyl L cysteine (FIG. 16C), and procainamide (FIG. 16D) for water-free spectra. Specifically each of the graphs in FIGS. 16A-16D compares calibration vectors obtained by training in the presence of an interferent, to the calibration vector obtained by training on clean plasma spectra alone. Large values (whether positive or negative) of the calibration vector generally represent wavelengths for which the corresponding spectral absorbance is sensitive to the presence of glucose, while small values of the calibration vectors generally represent wavelengths for which the spectral absorbance is insensitive to the presence of glucose. In the presence of an interfering substance, this correspondence is somewhat less transparent, being modified by the tendency of interfering substances to mask the presence of glucose.

FIGS. 16C and 16D show that in Example 3 there is substantial similarity between the calibration vectors computed by training on the interferent (n-acetyl L cysteine in FIG. 16C and procainamide in FIG. 16D) and by training on clean plasma alone. This similarity may reflect the fact that these two interferents are spectrally quite distinct from the glucose spectrum in the mid-infrared. FIGS. 16A and 16B show that in Example 3 there are relatively large differences between the calibration vectors calculated by training on the interferents mannitol (FIG. 16A) and dextran (FIG. 16B) and the calibration vectors obtained for clean plasma. These differences may represent a large degree of similarity between the spectra of these interferents and the spectrum of glucose in the mid-infrared region. Accordingly, FIGS. 16A-16D demonstrate that for those interferents having a spectrum that is similar to the glucose spectrum (e.g., mannitol and dextran), there may be a significant difference between the calibration vectors computed by training on the interferent and training on plasma alone. Also, if the interferent spectrum is substantially the same as the glucose spectrum (e.g., n-acetyl L cysteine and procainamide), there may be only relatively small differences between the calibration vectors obtained with and without the interferent.

### Likelihood-Weighted Methods for Interferent Determination

Additional methods for determining the concentration of an analyte in the presence of possible interferents include combining single interferent estimates of analyte concentrations. This type of method is referred to herein, without limitation, as a “likelihood-weighted average” approach. If no interferents are identified as possible interferents, any of the herein described methods may be used to determine analyte concentration.

With reference to the flowchart 400 shown in FIG. 4, one alternative embodiment performs the methods of Blocks 410 and 420 to obtain a sample measurement and to identify possible interferents. For the method of generating a model for predicting the analyte concentration for the obtained measurement (Block 430), certain embodiments perform the following: (a) determining the likelihood of possible interferent being present (e.g., being a probable interferent) and (b) for each of the probable interferents, estimating an analyte concentration in the presence of only that interferent (a “single interferent estimate”). For the method of applying the generated model to estimate an analyte concentration from the obtained measurement (Block 440), certain embodiments perform the following: (a) generating a weighting function for each of the possible interferents, and (b) combining the single interferent estimates for each possible interferents from Block 430 and the weighting function to generate a weighted average analyte estimation. Various alternative embodiments for Blocks 420 and 430 for an example likelihood-weighted average approach are described further below.

### Example Tests for Determining the Presence of Probable Interferents

In Block 420, the system may use one or more statistical and/or logical tests for determining possible interferents that are likely to be present in the sample obtained in Block 410. One or more tests may be used, singly or in combination, to identify probable interferents. A list of probable interferents may include none, one, some, or all of the interferents in the Library of Interferents.

In an embodiment of a first test (Test 1), if in Block 420 the system determines that an interferent (hereinafter denoted by ξ) is present at a level corresponding to a negative concentration, the system may interpret the negative concentration as a non-physical result and may exclude the possible interferent ξ from the list of probable interferents. In other embodiments, a negative concentration does not represent a non-physical result and indicates that the interferent in the obtained sample is at a concentration below the baseline value in the Sample Population. Accordingly, in some embodiments, a minimum interferent concentration (which may be zero or a negative value) is set, and a possible interferent is excluded from the list of probable interferents if its concentration is determined to be below the minimum interferent concentration.

In an embodiment of a second test (Test 2), the system computes the M 2 score for the interferent, for example, using Equation (1). In this embodiment, if the minimum MD² score is too high, then it is likely that the interferent ξ is not actually present or is not present in a large enough concentration to modify the analyte concentration estimate. The threshold MD² score used in this step may be empirically determined. For example, in one embodiment, it is found that a threshold value for the MD² score is in a range from about 50 to about 200. In other embodiments, the threshold MD² score is determined from a statistical level such as, e.g., the 95% quantile discussed with reference to FIGS. 6A and 6B.

In an embodiment of a third test (Test 3), a probability density that combines a range of probable interferent concentrations and the MD² score for that interferent is calculated. The probability density ρ(T) may be computed as a product of two probability densities:

ρ(T)=ρ_{χ}_{²}(MD²(Cₛ−Tξ))ρ_{T}(T), Eq. (2)

In one embodiment, the two probability densities are (1) the χ² distribution with N degrees of freedom (where N is the number of wavelengths present in the spectral measurements, for example 25), evaluated at the Mahalanobis score for the difference spectrum Cₛ′(T)=Cₛ−Tξ, and (2) the distribution of concentrations T for the interferent ξ. In some implementations, interferent concentration T is assumed to have a log-normal distribution with a value of the 95% quantile set at the assumed maximum interferent concentration Tₘₐₓ in the sample and a standard deviation of one half the mean. Other probability distributions may be used in other embodiments.

An integral of ρ(T) may then be computed over a range of possible interferent concentrations to determine a “raw probably score” (RPS). The range of the integral may, for example, be a semi-infinite range from 0 to infinity or a finite range, such as, e.g., from T_{MIN}=½T_{OPT} to T_{MAX}=2T_{OPT}. In an embodiment of Test 3, probable interferents ξ are selected to include those interferents having an RPS greater than a minimum value Pₘᵢₙ. The value of Pₘᵢₙ may be empirically determined from an analysis of the measurements. For example, a value of 0.70 may result in selection of a single possible interferent (a “single interferent identification”), and a value of 0.3 may give three probable interferents (a multiple interferent identification).

In certain embodiments, one or more of Test 1, Test 2, and Test 3 are utilized. For example, in an embodiment, the list of probable interferents ξ include those interferents from the Library that pass Test 1, Test 2, and Test 3. In some embodiments using multiple tests, later tests are performed only on those interferents ξ that pass all of the preceding tests. For example, Test 2 is applied only to interferents that pass Test 1, and Test 3 is applied only to those interferents that pass Test 2 (which of course have also passed Test 1 in an earlier step). Such embodiments advantageously may improve the computational performance of the method because the later, possibly more computationally burdensome tests (e.g., Test 3) are applied to a smaller subset of interferents than are present in the entire Library. In other embodiments, additional or different tests may be performed to identify the list of probable interferents.

In some embodiments, each test is applied in a serial fashion to each interferent ξ in the Library of Interferents, until the interferent ξ either fails a test or passes all the tests. In other embodiments the tests are applied in a parallel fashion to all possible interferents. For example, a first test is applied to all the interferents in the Library. A second test is then applied to all the interferents that pass the first test, and similarly for any further tests. In other embodiments, a combination of the serial and parallel approaches is used. In certain embodiments, the list of probable interferents includes all the interferents ξ that pass all the tests. In other embodiments, the list of probable interferents includes a subset of the interferents that pass the tests, for example, the 5, 10, or 20 most probable interferents. In another embodiment, the list of probable interferents includes only the single most probable interferent based on one or more statistical tests such as described above. In other embodiments, the list may include one (or more) interferents that are identified with the highest precision or accuracy. The number of interferents included on the list of probable interferents may be selected to reduce computational processing burden, to improve accuracy or precision of analyte estimation, and so forth.

### Example Single Interferent Calibration

An alternative embodiment of the actions performed in Block 430 may be used to calculate an analyte concentration in the presence of each possible interferent. In certain embodiments, the methods of alternate Block 430 are generally similar to the methods previously described with reference to FIG. 7, except as discussed below.

In some embodiments, the methods of Blocks 710 through 760 are performed for each possible interferent ξ, one at a time, resulting in an estimated single interferent calibration coefficient that is then used to generate a single interferent analyte concentration, denoted by g₁(ξ).

For example, in Block 710, the system may generate synthesized Sample Population spectra by adding a random concentration of interferent ξ to form an IESD. In Block 720 the system may partition the IESD into a calibration set and a test set. In Block 730 the system uses the calibration set to generate a calibration coefficient for predicting the analyte concentration in the presence of the interferent ξ. In Block 740 the system may estimate the analyte concentration in the test set in the presence of the interferent ξ. The error in the estimate is then calculated in Block 750. In some embodiments, Blocks 720 through 750 may be repeated to obtain estimates of the calibration coefficient and the error for different combinations of calibration sets and test sets. In Block 760 an average single interferent calibration coefficient, κ₁₋ₐᵥₑ(ξ) is calculated for the interferent ξ.

In some embodiments, the system applies each single interferent calibration κ₁₋ₐᵥₑ(ξ) to the measured spectra Cₛ to estimate a single interferent analyte concentration g₁(ξ). The single interferent analyte concentration may be calculated according to g₁(ξ)=κ₁₋ₐᵥₑ(ξ) Cₛ.

### Example Likelihood-Weighted Analyte Estimation

In some embodiments, the system generates a weighting function p(ξ) for each of the possible interferents ξ and combines the single interferent estimates and the weighting functions to generate a weighted average analyte estimation.

For example, in certain embodiments, the raw probability score (RPS) determined in Block 420 is rescaled to unit probability to give a weighting function p(ξ) that can be used for each probable interferent. In one embodiment, the weighting function for the interferent ξ equals the RPS for the interferent ξ divided by the sum of the RPSs for all the probable interferents, p(ξ)=RPS(ξ)/ΣRPS(ξ). In other embodiments, the weighting function is the same constant for each interferent ξ (e.g., p(ξ)=1/(number of probable interferents)). In yet other embodiments, the weighting functions are chosen to be inversely proportional to the errors in the single interferent analyte concentration (e.g., p(ξ)∝1/rms²).

In some embodiments, the system combines the weighting functions and the single interferent analyte concentrations into a “likelihood-weighted” average concentration, g, according to:

g=Σg₁(ξ)p(ξ), Eq. (3)

where the sum is over all interferents ξ on the list of probable interferents. In implementations where the weighting functions p(ξ) are the same constant value for all interferents, the likelihood-weighted average concentration is the ordinary arithmetic average of the single interferent concentrations. These embodiments of the method are called “likelihood-weighted single-interferent rejection” methods and denoted by “LW1IF.”

As described above, in certain embodiments the single interferent analyte concentration is determined as g₁(ξ)=K₁₋ₐᵥₑ(ξ)Cₛ. Accordingly, Equation (3) shows that in certain such embodiments the likelihood-weighted average analyte concentration may be determined as g=κCₛ, where κ=Σₚ(ξ)κ₁₋ₐᵥₑ(ξ), where the sum is over all interferents ξ on the list of probable interferents. Thus, in these embodiments, the calibration coefficient κ that may be applied to the sample measurement (e.g., the spectrum Cₛ) is a weighted average of the single interferent calibrations K₁₋ₐᵥₑ(ξ).

In some embodiments, only the single most probable interferent is used to determine the analyte concentration. In such embodiments, only the most likely interferent from the list of probable interferents is used in the analysis. The most likely interferent may be selected to be the interferent ξ that maximizes a single probability metric. Such embodiments of the disclosed methods are called “maximum-probability single-interferent rejection” methods and denoted by “MP1IF.” Possible advantages of certain MP1IF methods include computational speed (since only a single interferent is used) and relative simplicity of programming.

### Example 4

The “likelihood-weighted average” approach has been tested via simulated data (e.g., spectra generated as the sum of clean blood plasma spectra and random concentrations of interferent spectra) as well as spectra coming from plasma obtained from an intensive care unit. Example 4 to be described in detail below compares an embodiment of the likelihood-weighted single-IF rejection method (LW1IF) with an embodiment of the maximum-probability single-IF rejection (MP1IF) method.

Ten thousand test spectra were generated, each containing random amounts of up to six interfering substances at concentrations randomly chosen from log-normal distributions. The statistical parameters of the log-normal distribution were selected based on interferent concentrations deemed likely to occur in the plasma samples. The 95th percentile of the log-normal distribution was placed at the (published) maximum concentration level, and the standard deviation was set at one-half the mean value for the distribution.

Of the 10000 test spectra, the system determined that a set of 4537 spectra passed the tests described above with reference to Block 420 for single interferent rejection. Of this set, 2590 spectra had an MD² score indicating that no correction to analyte concentration was needed. The remaining 1947 spectra had an MD² score that passed the single-interferent test criteria. In Example 4, the population of spectra that passed the criteria of Test1, Test2, and Test 3 was broader than expected for the MP1IF method, in which the Pₘᵢₙ threshold was 0.75 (as compared to 0.30 in the present test) in order to function as well. In the simulated population described here, many spectra contain more than a single interferent as shown in the following Table 4.

TABLE 4
Numbers of interferents present among
4537 spectral samples in Example 4

| # Interferents | # Spectra |
|---|---|
| 0 | 1437 |
| 1 | 1190 |
| 2 | 846 |
| 3 | 543 |
| 4 | 304 |
| 5 | 150 |
| 6 | 67 |

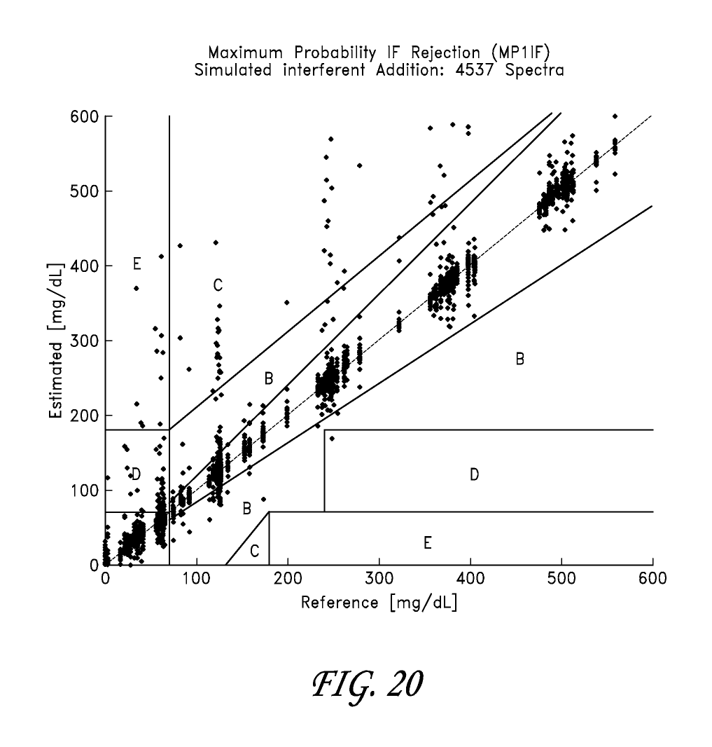

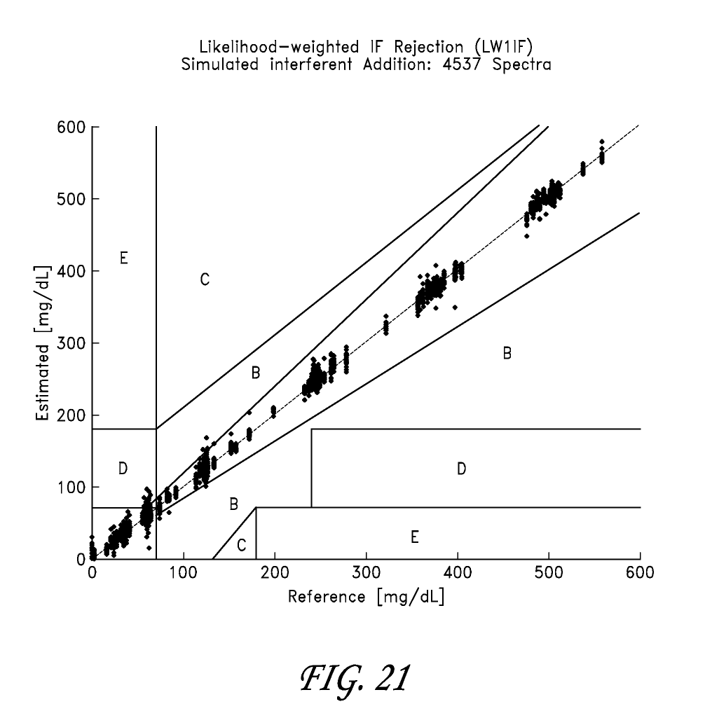

FIGS. 20, 21, and 22 compare the performance of the above-described embodiments of the MP1IF and LW1IF techniques. FIGS. 20 and 21 show (on Clarke error grids) the measured (reference) and estimated glucose values for the 4537 samples. FIG. 20 shows estimated glucose concentrations (in mg/dL) using the example MP1IF technique, and FIG. 21 shows estimated glucose concentrations using the example LW1IF technique. A comparison of the scatter of the estimates in FIG. 21 (LW1IF) compared to the scatter in FIG. 20 (MP1IF) shows that glucose estimates with the example LW1IF technique may provide a much tighter distribution of errors. Statistical analysis of the data presented in FIGS. 20 and 21 demonstrates a bias of 4.2 mg/dL and a standard deviation of error of 31.6 mg/dL for the example MP1IF technique compared to a bias of 0.15 mg/dL and a standard deviation of error of 6.4 mg/dL for the example LW1IF technique.

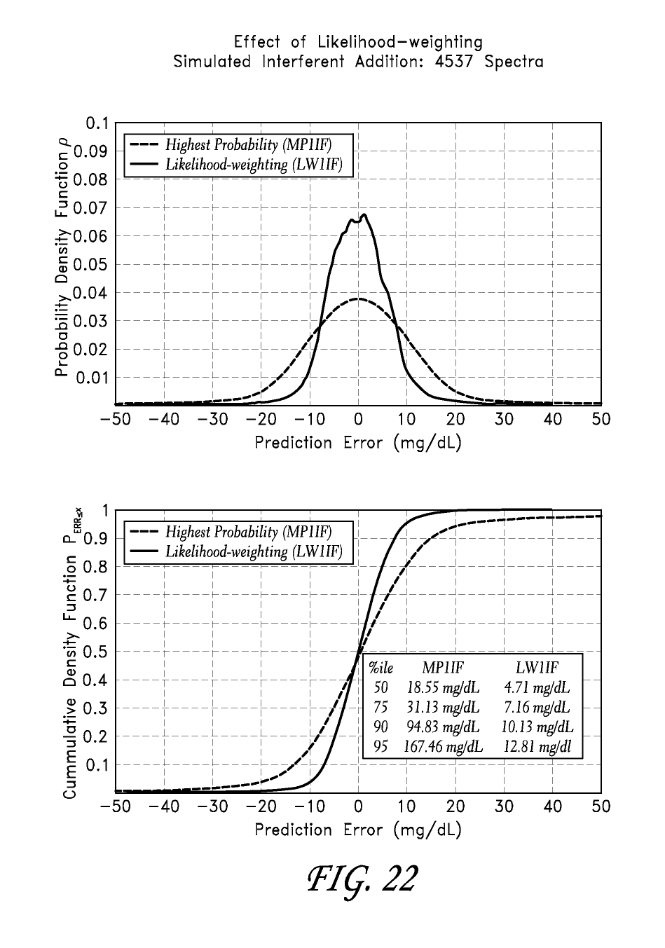

The difference in scatter apparent in FIGS. 20 and 21 between the glucose estimates determined from the example MP1IF and LW1IF techniques is shown quantitatively in FIG. 22. The upper panel illustrates probability density functions, and the lower panel illustrates cumulative probability functions corresponding to the probability density functions in the upper panel. The lower panel also includes a table that lists percentiles for absolute error. based on the probability functions shown in FIG. 22. The data in FIG. 22 demonstrate that the probability density function for prediction error is substantially narrower for the example LW1IF technique than the example MP1IF technique.

It will be understood that the steps of methods discussed herein may be performed by an appropriate processor (or processors) of a processing (e.g., computer) system executing software instructions (e.g., code segments) stored in appropriate storage. The processors may be on the same or different physical machines. The processors may include general and/or special purpose components. The software instructions may be stored as computer-executable instructions on any form of computer-readable medium. It will also be understood that the disclosed methods and apparatus are not limited to any particular implementation, programming language, and/or programming technique and that the methods and apparatus may be implemented using any appropriate techniques for implementing the functionality described herein. The methods and apparatus are not limited to any particular programming language or operating system. In addition, the various components of the apparatus may be included in a single housing or in multiple housings that communication by wired and/or wireless communication.

Further, the interferent, analyte, or population data used in the method may be updated, changed, added, removed, or otherwise modified as needed. Thus, for example, spectral information and/or concentrations of interferents that are accessible to the methods may be updated or changed by updating or changing a database of a program implementing the method. The updating may occur by providing new computer readable media or over a computer network. Other changes that may be made to the methods or apparatus include, but are not limited to, the adding of additional analytes or the changing of population spectral information.

One embodiment of each of the methods described herein may include a computer program accessible to and/or executable by a processing system, e.g., a one or more processors and memories that are part of an embedded system. Thus, as will be appreciated by those skilled in the art, embodiments of the disclosed inventions may be embodied as a method, an apparatus such as a special purpose apparatus, an apparatus such as a data processing system, or as a carrier medium, e.g., a computer program product. The carrier medium carries one or more computer readable code segments for controlling a processing system to implement a method. Accordingly, various ones of the disclosed inventions may take the form of a method, an entirely hardware embodiment, an entirely software embodiment or an embodiment combining software and hardware aspects. Certain aspects of the disclosed methods and systems may be embodied as firmware. Furthermore, any one or more of the disclosed methods (including but not limited to the disclosed methods of measurement analysis, interferent determination, and/or calibration coefficient generation) may be stored as one or more computer readable code segments or data compilations on a carrier medium. Any suitable computer readable carrier medium may be used including a magnetic storage device such as a diskette or a hard disk; a memory cartridge, module, card or chip (either alone or installed within a larger device); or an optical storage device such as a CD or DVD.

Reference throughout this specification to “one embodiment” or “an embodiment” means that a particular feature, structure or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to one of ordinary skill in the art from this disclosure, in one or more embodiments.

Similarly, it should be appreciated that in the above description of example embodiments, various features of the inventions are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, as the following claims reflect, inventive aspects lie in a combination of fewer than all features of any single foregoing disclosed embodiment. Thus, the claims following the Detailed Description are hereby expressly incorporated into this Detailed Description, with each claim standing on its own as a separate embodiment.

Further information on analyte detection systems, sample elements, algorithms and methods for computing analyte concentrations, and other related apparatus and methods can be found in U.S. Patent Application Publication No. 2003/0090649, published May 15, 2003, titled REAGENT-LESS WHOLE BLOOD GLUCOSE METER; U.S. Patent Application Publication No. 2003/0178569, published Sep. 25, 2003, titled PATHLENGTH-INDEPENDENT METHODS FOR OPTICALLY DETERMINING MATERIAL COMPOSITION; U.S. Patent Application Publication No. 2004/0019431, published Jan. 29, 2004, titled METHOD OF DETERMINING AN ANALYTE CONCENTRATION IN A SAMPLE FROM AN ABSORPTION SPECTRUM; U.S. Patent Application Publication No. 2005/0036147, published Feb. 17, 2005, titled METHOD OF DETERMINING ANALYTE CONCENTRATION IN A SAMPLE USING INFRARED TRANSMISSION DATA; and U.S. Patent Application Publication No. 2005/0038357, published on Feb. 17, 2005, titled SAMPLE ELEMENT WITH BARRIER MATERIAL. The entire contents of each of the above-mentioned publications are hereby incorporated by reference herein and are made a part of this specification.

A number of applications, publications and external documents are incorporated by reference herein. Any conflict or contradiction between a statement in the bodily text of this specification and a statement in any of the incorporated documents is to be resolved in favor of the statement in the bodily text.

What is claimed is:

1. A method for estimating a concentration of an analyte in a sample from a measurement of the sample, the method comprising:
determining, based on the measurement, a list of one or more possible interferents to the measurement of the analyte in the sample;
calculating, for each one of the possible interferents on the list, a single interferent analyte concentration based on the presumed presence in the sample of the one interferent and no other interferents from the list; and
determining an estimated analyte concentration based at least in part on the single interferent analyte concentrations.

2. The method of claim 1, wherein the list includes all the interferents in a library of interferents.

3. The method of claim 1, wherein the list includes a single interferent.

4. The method of claim 3, wherein the single interferent is the most probable interferent.

5. The method of claim 1, wherein determining the list of one or more possible interferents comprises performing a statistical test for the presence of the one or more possible interferents.

6. The method of claim 5, wherein the statistical test comprises determining information related to a Mahalanobis distance.

7. The method of claim 5, wherein the statistical test comprises comparing an estimated concentration of a possible interferent to a threshold concentration.

8. The method of claim 1, wherein determining a list of possible interferents comprises analyzing combinations of sample spectra and interferent spectra corresponding to varying combinations of a selected interferent and identifying the selected interferent as a possible interferent if any of the combinations are within predetermined bounds.

9. The method of claim 1, wherein calculating a single interferent analyte concentration comprises:
determining a calibration which reduces error attributable to the presumed presence in the sample of the one interferent;
applying the calibration to the measurement; and
estimating, based on the calibrated measurement, the single interferent analyte concentration.

10. The method of claim 9, wherein calculating a single-interferent analyte concentration further comprises determining, for each one of the interferents on the list, a weighting function, and wherein determining an estimated analyte concentration comprises combining the single interferent analyte concentrations and the weighting functions.

11. The method of claim 10, wherein the weighting function for an interferent is based at least in part on a probability of the presence of the interferent in the sample.

12. The method of claim 1, wherein the measurement comprises a spectrum.

13. The method of claim 1, wherein the measurement comprises an infrared spectrum.

14. The method of claim 1, wherein the sample includes at least one component of blood, and wherein the analyte comprises glucose.

15. The method of claim 1, wherein the sample comprises a bodily fluid and the list includes at least one exogenous interferent.

16. A carrier medium carrying one or more computer readable code segments to instruct a processor to implement a method for estimating the amount of an analyte in a sample from a measurement of the sample, the method comprising the method of claim 1.

17. An apparatus for estimating a concentration of an analyte in a sample from a measurement of the sample, the apparatus comprising:
means for determining, based on the measurement, a list of one or more possible interferents to the measurement of the analyte in the sample;
means for calculating, for each one of the interferents on the list, a single interferent analyte concentration based on the presumed presence in the sample of the one interferent and no other interferents from the list; and
means for determining an estimated analyte concentration based at least in part on the single interferent analyte concentrations.

18. The apparatus of claim 17, wherein the means for determining a list of possible interferents comprises:
means for analyzing combinations of sample spectra and interferent spectra corresponding to varying combinations of a selected interferent; and
means for identifying the selected interferent as a possible interferent if any of the combinations are within predetermined bounds.

19. The apparatus of claim 17, wherein the means for calculating a single interferent analyte concentration comprises:
means for determining a calibration which reduces error attributable to the presumed presence in the sample of the one interferent;
means for applying the calibration to the measurement; and
means for estimating, based on the calibrated measurement, the single interferent analyte concentration.

20. The apparatus of claim 17, further comprising means for outputting information related to the estimated analyte concentration.

21. The apparatus of claim 17, wherein the sample includes at least one component of blood, and wherein the analyte comprises glucose.

22. An analyte detection system comprising:
a sensor system configured to provide information relating to a measurement of an analyte in a sample; and
a processor system configured to execute stored program instructions such that the analyte detection system:
determines, based on the measurement, a list of one or more possible interferents to the measurement of the analyte in the sample;
calculates, for each one of the interferents on the list, a single interferent analyte concentration based on the presumed presence in the sample of the one interferent and no other interferents from the list; and
determines an estimated analyte concentration based at least in part on the single interferent analyte concentrations.

23. The analyte detection system of claim 22, wherein the sensor system comprises a spectroscope and the measurement comprises a spectrum.

24. The analyte detection system of claim 23, wherein the sample comprises at least one component of blood and the analyte comprises glucose.

25. The analyte detection system of claim 22, wherein the sample comprises a bodily fluid, and wherein the list includes at least one exogenous interferent.

26. The analyte detection system of claim 22, further comprising a source of electromagnetic radiation, wherein the sensor system comprises a detector configured to detect radiation emitted by the source that interacts with the sample.

27. The analyte detection system of claim 26, wherein at least a portion of the radiation emitted by the source is transmitted through the sample and detected by the detector.

28. The analyte detection system of claim 22, wherein the sensor system is physically remote from the processor system.

29. The analyte detection system of claim 28, wherein the sensor system is configured to communicate with the processor system via a network.

30. The analyte detection system of claim 29, wherein the network comprises a hospital information network.

31. The analyte detection system of claim 22, further comprising a user interface configured to output information related to the estimated analyte concentration.

32. The analyte detection system of claim 22, wherein the processor system is configured to execute stored program instructions such that the analyte detection system determines a list of possible interferents by analyzing combinations of sample spectra and interferent spectra corresponding to varying combinations of a selected interferent and identifying the selected interferent as a possible interferent if any of the combinations are within predetermined bounds.

33. The analyte detection system of claim 22, wherein the processor system is configured to execute stored program instructions such that the analyte detection system determines a list of possible interferents by performing a statistical test for the presence of the one or more possible interferents.

34. The analyte detection system of claim 33, wherein the list comprises a single most probable interferent.

35. The analyte detection system of claim 22, further comprising a sampling system configured to draw the sample from a patient periodically and to transport the sample into operative engagement with the sensor system.
